(19) United States
(12) Patent Application Publication
(10) Pub. No.: US 2014/0378526 A1
(43) Pub. Date: Dec. 25, 2014

(54) DESIGN OF NUCLEIC ACID BINDING MOLECULES WITH NON-WATSON CRICK AND NON-CANONICAL PAIRING BASED ON ARTIFICIAL MUTATION CONSENSUS SEQUENCES TO COUNTER ESCAPE MUTATIONS

(71) Applicant: CITY OF HOPE, Duarte, CA (US)

(72) Inventors: John J. ROSSI, Duarte, CA (US); Ulrike JUNG, Duarte, CA (US)

(21) Appl. No.: 14/309,803

(22) Filed: Jun. 19, 2014

Related U.S. Application Data
(63) Continuation-in-part of application No. 13/842,977, filed on Mar. 15, 2013.
(60) Provisional application No. 61/646,181, filed on May 11, 2012.

(51) Int. Cl. C12N 15/113; C12Q 1/70

(52) U.S. Cl. CPC C12N 15/1132; C12Q 1/703; C12N 2320/30; C12N 2330/31; C12N 2310/14; C12Q 2600/178; C12Q 2600/136
USPC 514/44 A; 536/24.5; 536/23.1; 435/5; 506/9

(57) ABSTRACT

Universal nucleic acid binding molecules (e.g., antisense oligonucleotides or RNAi molecules) having an inhibitory or activating nucleic acid sequence which binds a receiving nucleic acid sequence (e.g., RNA or DNA) are provided. In some embodiments, the universal nucleic acid binding molecules bind the receiving nucleic acid sequence (e.g., RNA or DNA) via at least one non-Watson Crick or non-canonical paired base.

### PRIORITY CLAIM

This application claims priority of U.S. Provisional Application No. 61/646,181, filed May 11, 2012, and is a continuation-in-part of U.S. application Ser. No. 13/842,977, filed Mar. 15, 2013; the subject matter of both of which is hereby incorporated by reference as if fully set forth herein.

### STATEMENT OF GOVERNMENT INTEREST

The present invention was made with government support under Grant Nos. HL07470, AI392391 and AI42552, awarded by the National Institutes of Health. The Government has certain rights in the invention.

### BACKGROUND

RNA interference (RNAi) is a mechanism endogenously existing in cells to suppress the expression of genes through interaction of protein complexes with the mRNA. Naturally occurring RNAi relies on microRNAs. MicroRNAs (miRNA) are processed from RNAs sequences that fold into an internal stem-loop structure. 5′ and 3′ adjacent sequences are cut off as well as the loop leaving a short double stranded RNA with usually 2 nucleotides overhangs on the 3′ end of each strand. One of the strands is degraded while the other strand remains (the guide strand). The guide strand is incorporated into a protein complex leading to degradation or translational repression of mRNA targets, which are at least partially complementary. The mechanism of RNAi has been artificially employed since several years now by the introduction of shRNA stem-loop sequences that are processed like miRNA but due to full complementarity with the target mRNA always leads to its degradation. And there are siRNAs that are used as triggers of RNAi, which are already fully processed miRNA but usually contain full complementary strands and show full complementarity with the target RNA the same as processed shRNAs.

In addition to RNAi mechanisms, post-transcriptional or translational repression of mRNA targets may also be accomplished by antisense technology. In this case, single-stranded antisense oligonucleotides may be used to suppress the expression of a target gene.

RNAi- or antisense-based therapies are very appealing due to their target specificity at very low doses, which can avoid common drug intrinsic side effects. Furthermore, in case of long term treatment like gene therapy, RNA and other nucleic acid molecules have the advantage to be not prone to immune response while proteins are.

However, current RNAi molecules (e.g., siRNAs and shRNAs (collectively, “si/shRNAs”)) and antisense oligonucleotides targeting mammalian genes or RNAs (like virus genomes for example) are designed based on actual sequences occurring in nature. In case of viruses that mutate the target sequence, it relies on the identification of conserved regions in naturally available strain sequences. But, even conserved regions are not without mutations and the therapeutic pressure of therapeutic si/shRNAs or antisense oligonucleotides readily leads to escape variants which render these si/shRNAs or antisense oligonucleotides ineffective. Prime examples to generate escape strains are Human Immunodeficiency virus (HIV), Hepatitis C virus (HCV) and Hepatitis B virus (HBV). Therefore, it would be desired to generate universal nucleic acid binding molecules (e.g., antisense oligonucleotides and/or RNA interference molecules) that would retain their efficacy when such viruses mutate.

### SUMMARY

Universal nucleic acid binding molecules (e.g., antisense oligonucleotides or RNAi molecules) having an inhibitory or activating nucleic acid sequence which binds a receiving nucleic acid sequence (e.g., RNA or DNA) are provided herein. In some embodiments, the universal nucleic acid binding molecules bind the receiving nucleic acid sequence (e.g., RNA or DNA) via at least one non-Watson Crick or non-canonical paired base. In some embodiments, the receiving nucleic acid sequence (e.g., RNA or DNA) is a target viral RNA sequence derived from a human immunodeficiency HIV virus, a hepatitis B virus (HBV), a hepatitis C virus (HCV), or an influenza virus.

In some embodiments, methods for treating a subject having a disease or condition (e.g., a subject infected with a target virus or a subject having a chemotherapy-resistant cancer) are provided. Such embodiments include a step of administering a therapeutically effective amount of a universal nucleic acid binding molecule which comprises an inhibitory or activating nucleic acid sequence which binds a receiving nucleic acid sequence via at least one non-Watson Crick or non-canonical paired base. In some aspects the inhibitory or activating nucleic acid sequence activates or suppresses the expression or activity of a target molecule.

In one aspect, a receiving nucleic acid sequence may be a viral RNA sequence derived from a human immunodeficiency HIV virus. In such embodiments, the viral RNA sequence may include an IUPAC sequence of URYCARUAYAUGGAYGAYYURUAUGURGG (SEQ ID NO:1) or YURGAYACRGGRGCAGAUGAUACAGUR (SEQ ID NO:2). In some aspects, the inhibitory or activating nucleic acid sequence may include an IUPAC or nucleotide sequence of YARRTCRTCCATRTAYTGRYA (SEQ ID NO:3); TAYARRTCRTCCATRTAYTGR (SEQ ID NO:4); TAGGTCGTCCATGTATTGGTA (SEQ ID NO:5); TATAGGTCGTCCATGTATTGG (SEQ ID NO:6); YACTGTATCATCTGCYCCYGT (SEQ ID NO:7); YADYACTGTATCATCTGCYCC (SEQ ID NO:8); DYACTGTATCATCTGCYCCYG (SEQ ID NO:9); ADYACTGTATCATCTGCYCCY (SEQ ID NO:10); TACTGTATCATCTGCTCCTGT (SEQ ID NO:11); TAGTACTGTATCATCTGCTCC (SEQ ID NO:12); GTACTGTATCATCTGCTCCTG (SEQ ID NO:13); or AGTACTGTATCATCTGCTCCT (SEQ ID NO:14).

In some embodiments, methods of designing a universal nucleic acid binding molecule are provided, and may include steps of generating an inhibitory or activating nucleic acid sequence by generating a consensus sequence derived from two or more receiving nucleic acid sequences of host or foreign origin and converting the consensus sequence into an International Union of Pure and Applied Chemistry (IUPAC) sequence code by randomly or selectively replacing U or G's with a corresponding ambiguous IUPAC coded base (Y or R). The methods may alternatively include a step of generating an artificial target sequence from one target nucleic acid sequence of host or foreign origin by randomly or selectively replacing U or G's with a corresponding ambiguous IUPAC coded base (Y or R). The methods may also include a step of substituting each corresponding ambiguous base with a base that allows for Watson-Crick, non-Watson-Crick, and/or non-canonical pairings. One or more inhibitory or activating nucleic acid molecules may then be selected using any suitable selection methods including, but not limited to, an RNAi selection method, an antisense oligonucleotide selection protocol, or an RNA-based transcriptional activation or inhibition selection method. In some aspects, the consensus sequence is derived from a plurality of clinical isolates.

### BRIEF DESCRIPTION OF THE DRAWINGS

FIG. 1A shows the origin of HIV subtypes and subtype composition selected for development of IUPAC artificial mutant sequence according to some embodiments. 54 strains were selected from the most densely populated or geographically widely distributed areas in the world (shaded countries) to cover as many HIV-1 subtypes as possible and to ensure maximum strain variance. Several subtypes are represented in the selected strains, which are as indicated.

FIG. 1B shows the selection of target sites (region 4 and region 3) in the HIV gag-pol gene using MuSTER design rules, resulting in target site in the Gag-Pol polyprotein mRNA that is downstream of a partially active ribosome frameshift.

FIGS. 1C and 1D shows the two selected target regions in the HIV gag-pol gene based on the location and proportion of guide strand positions without Watson/Crick pairing.

FIG. 2 shows knockdown of Renilla luciferase normalized to Firefly luciferase in psiCheck2.1 constructs with 1056 nucleotides of integrated gag-pol region according to some embodiments.

FIG. 3 shows transfection control between different samples according to some embodiments. Firefly absolute values 48 hours after transfection vary little and show no correlation with the knockdown values obtained. Mean of technical triplicate with average deviation.

FIG. 4 shows a standard curve p24 assay pNL4-3/HlfB according to some embodiments.

FIG. 5 shows p24 Levels relative to sample transfected just with empty (no shRNA) construct according to some embodiments. HlfB cells were transfected with Lipofectamine 2000 and each sample received 100 ng pCMV-rev plasmid and 700 ng of a plasmid mix containing 338 ng shRNA or empty construct (a) with the remainder comprised of empty construct or 26 ng shRNA or empty construct (b) with the remainder comprised of empty construct.

FIG. 6: p24 Levels relative to sample transfected just with empty (no shRNA) construct according to some embodiments. HEK293T cells were transfected with Lipofectamine 2000 and each sample received 200 ng pNL4-3 plasmid and 600 ng of a plasmid mix containing 338 ng shRNA or empty construct with the remainder comprised of empty construct.

FIG. 7 illustrates the activity of MuSTER and conventional shRNA (U6 driven or MuSTER intron background) in IIIB challenge of CEM-CCR5 cells stably transduced according to some embodiments. Empty is vector without any anti-HIV shRNA.

FIG. 8 shows p24 levels in CEM-CCR5 cells transduced with lentivirual MuSTER-designed expression constructs and challenged with the HIV IIIB strain, normalized to an empty construct (day 8) according to some embodiments. S1 is a conventional anti-HIV from the same cassette or U6 promoter driven. MuSTER-designed shRNA constructs Mu3-2, Mu3-3, Mu4-1, Mu4-2, Mu4-3 and Mu4-4 are the same as, and correspond to, shRNA constructs R3-2, R3-3, R4-1, R4-2, R4-3 and R4-4 described herein.

FIG. 9 shows the impact of target RNA sequence mutation inside and outside of the target RNA sequence region on the knockdown ability of MuSTER-designed shRNA constructs according to some embodiments.

FIG. 10 shows the details of the shRNA positions and binding regions of the constructs shown in FIG. 9 according to some embodiments.

FIG. 11 shows a p24 photography assay at day 8 (A), day 11 (B) and day 15 (C) post challenged of CEM CCR5 cells with HIV IIIB according to some embodiments. All samples diluted by 1:100.

FIG. 12 shows canonical and non-canonical pairings used in MuSTER shRNA design. (A) Canonical Watson/Crick and non-canonical RNA pairings and their H bonds according to Panigrahi et al. 2011 with IUPAC lettering. (B) Crystallographic structure of CGG/CUG RNA repeats yielding RNA A-form helix double strands (28), dark colors in each strand depict Guanine, light color in each strand Cytosine, GG pairing in 3rd and 5th base pair from the bottom

FIG. 13 is a Table showing the shRNA oligonucleotides produced in the examples below, according to some embodiments. All constructs were confirmed by sequencing.

FIG. 14 shows the loss of anti-HIV activity in p24 assays and Northern Blot upon MuSTER modification (incorporation of bulge structure position 9-11), which confirms RNAi as underlying mechanism in some embodiments. (A) Relative p24 values of two independent experiments 48 hours after co-transfection of RNAi vector plasmid and pNL4-3 into HEK 293T. (B) Northern Blot of total RNA purified from one experiment, Inf-CNTR: HIV expression in HeLa cell total RNA, Uninf-CNTR: untreated HeLa cell total RNA, 2 independent vector transfection for batch effect control, shPos targets the 5′ UTR of pNL4-3, MuSTER target the pol region found only in unspliced HIV RNA. Quality control for the Northern Blot RNA and intracellular shRNA expression/processing is shown in FIG. 18, below. US: unspliced RNA species, PS partially spliced, FS fully spliced.

FIG. 15 shows that MuSTER shRNAs inhibit a broad range of HIV-1 subtypes and outperform conventional shRNAs in packaging and challenge tests, according to some embodiments. (A) p24 assay from five different HIV strains 48 hours post co-transfection with conventional or MuSTER shRNA plasmids, 2 independent experiments. Values normalized to empty construct=100, dark red bar depicting mean p24. (B) p24 assay from CEM NKR CCR5 T cells stably expressing MuSTER cassettes after sorting, challenged with pool of HIV strains from A (Pool, day 16 post challenge) or HIV BAL III quasi-species (BAL, day 15 post challenge). Values normalized to conv. shRNA=100. Paired, two-sided t-test. FIGS. 15C and D show alignment of HIV strains for RNAi trigger targets (red shaded areas). (C) Target sequence of conventionally designed control shRNA. (D) Target region of MuSTER 3-6. MuSTER 3 is coincidentally in NL4-3 and SG3.1 a canonically binding shRNA while conv. shRNA S1 is the most potent conventional shRNA from certain clinical trials (see Li et al. 2005; DiGiusto et al 2010), which targets the 5′ UTR region of HIV mRNA.

FIG. 16 shows that MuSTER 5 restriction of HIV-1 mutant pool or quasispecies mixture is stable or improves over time compared to conventional design, according to some embodiments. p24 capture ELISA from supernatant of CEM NKR CCR5 T cells stably expressing MuSTER cassettes after sorting, challenged with HIV BAL III quasi-species or pool of HIV strains as depicted in FIG. 15. Values normalized to conv. shRNA=100.

FIG. 17 shows quality control of total RNA gel for FIG. 4 (A), and Northern Blot analysis of intracellular expression/processing of conventional shRNA (ShPos, or S1) and MuSTER shRNA and bulged controls (bul) from same samples (B), according to some embodiments. Northern analysis shows that the shRNAs are all expressed and correctly processed to the mature form. The detection is qualitative and not quantitative since northern probes, designed to be complementary to the bulged controls, have the ability to cross-hybridize with the corresponding active shRNAs. Some of the probes (MuSTER 6 & MuSTER 6 bul) can also self-pair reducing the detection of the matching shRNAs; thus efficiency of hybridization and signal strength cannot be accurately compared between samples. A γ-³²P labeled probe complementary to the 3′ end of the endogenous U6snRNA was used to verify loading and integrity of the RNA analyzed in each lane.

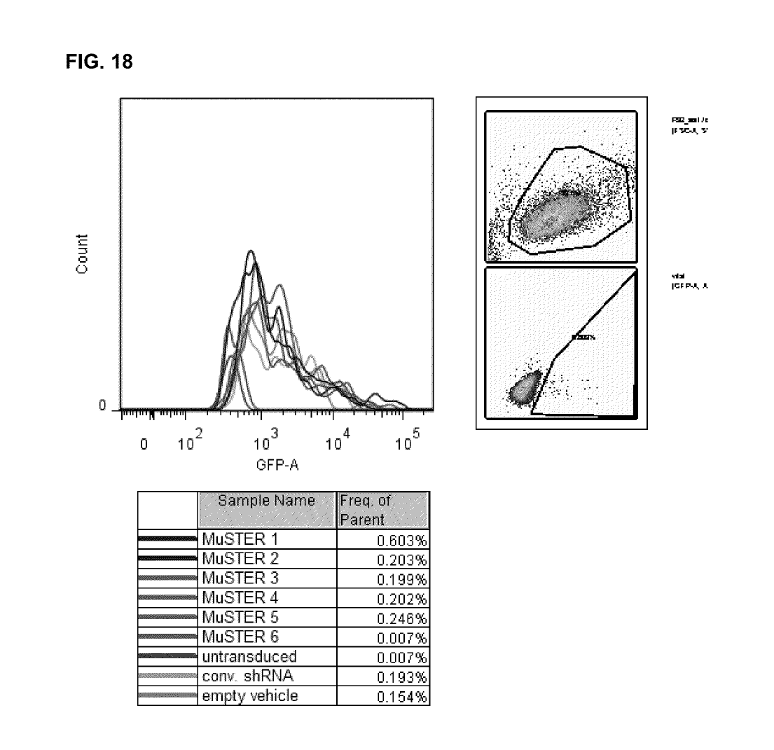

FIG. 18 shows OEM NKR CCR5GFP expression during sorting according to one embodiment. Cells were sorted for GFP (FL-1/first channel) against auto-fluorescence (FL-2/PE channel) after gating for cells with normal morphology.

FIG. 19 is a Python script to identify MuSTER rule compliant sequences from an IUPAC encoded target according to one embodiment. This module will search for certain letter combinations of up to IUPAC code (containing A/C/T/G/Y/R) in an consensus target sequence and print out all found substrings/subsequences with at least 16 nucleotides length. Output saves in output text file: target sequence candidate, length of target sequence candidate, position of target sequence candidate in consensus. IUPAC target consensus must be formatted into a single line in a plain text file and not contain any empty spaces or numbers. File format: IUPAC[ENTER button][target consensus sequence here]. The example shown in the figure with this script text is then copied into a text file named MuSTER_target_extract.py to create a script file.

FIG. 20 is a table of HIV-1 strains used in the alignment described herein.

### DETAILED DESCRIPTION

Universal nucleic acid binding molecules that modulate (i.e., suppress or activate) the expression and/or activity of one or more target molecules by binding a receiving nucleic acid sequence (e.g., a receiving RNA sequence or a receiving DNA sequence) via at least one non-Watson Crick or non-canonical paired base are provided herein. In accordance with the embodiments described herein, said universal nucleic acid binding molecules may include RNA interference (RNAi) molecules, antisense oligonucleotides, or any other nucleic acid molecules which act through a mechanism whereby expression of a gene or protein is suppressed or activated (e.g., by transcriptional or post-transcriptional action by siRNA or other molecules described herein). The vulnerability of common antisense or RNAi molecules such as antisense oligonucleotides, siRNA, and/or shRNA lies in the design based on a single natural target sequence—while the design rules generally allow only Watson-Crick pairing between guide strand and target. This makes current antisense and RNAi design protocols limited. Watson-Crick pairings are pairings between the bases Adenine and Uracil in RNA and Guanine and Cytosine. However, non-Watson Crick pairings and non-canonical pairings which have different energetic and spatial properties are possible too and have been found in natural RNA. The most common of these is the pairing between Uracil and Guanine. Others exist which include the non-conventional base Inosine to allow pairings between Inosine & Uracil, Inosine & Adenine and Inosine & Cytosine as well as the Uracil-Cytosin 4-carbonyl-amino pairing which has almost the same structure like the Uracil-Guanine pairing (Anderson, O'Neil et al. 1999). Pairing between modified or artificial bases are possible as well and can lead to even more effective binding than with naturally occurring bases (Sun, Sheng et al. 2012; Vendeix, Murphy et al. 2012).

Introducing non-Watson Crick or non-canonical pairing between an antisense oligonucleotide or an si/shRNA molecule and the target sequence may still lead to significant gene suppression or activation, and in some cases may even lead to increased effects in vivo as compared to fully complementary sequences. As such, designing universal nucleic acid binding molecules using non-Watson Crick and non-canonical pairing makes it possible to develop and design such molecules for use as therapeutic or research agents that, among other things may (i) bind one or more target nucleic acid sequences more efficiently or effectively than molecules that bind using only Watson Crick pairing; (ii) retain their therapeutic, physiological and/or other functional activity to a particular target molecule across species; and/or (iii) retain their therapeutic, physiological and/or other functional activity to a particular target molecule when the target molecule undergoes mutations (e.g., escape mutations). These characteristics allow researchers and clinicians to treat or test multiple conditions and/or multiple species using a single universal binding molecule.

According to some embodiments described herein, a universal nucleic acid binding molecule may be any suitable type of RNAi molecule which, when delivered to a target cell, associates with a RISC complex and binds a receiving nucleic acid (e.g., DNA or RNA) sequence to activate or inhibit (i.e., suppress or silence) the expression of one or more target molecules in the target cell via transcriptional or post-transcriptional activation or inhibition. RNAi molecules that may be used as a universal nucleic acid binding molecule in accordance with the embodiments described herein include, but are not limited to, micro RNA (miRNA) molecules, small interfering RNA (siRNA) molecules, and short hairpin RNA (shRNA) molecules. The RNAi molecule may be delivered to the target cell as a double-stranded RNAi molecule, or may be pre-separated into a single-stranded RNAi molecule prior to delivery, allowing the RNAi molecule to directly associate with the RISC complex. In certain aspects, the RNAi molecule binds a segment of a receiving RNA sequence produced by a coding or non-coding region of a particular gene or DNA sequence to post-transcriptionally activate or suppress expression of one or more target molecules. The receiving RNA sequence may be a protein-coding RNA (e.g., mRNA) or a non-protein-coding RNA (e.g., IncRNA, tRNA, miRNA, rRNA, snRNA and others). In other aspects, the RNAi molecule binds a segment of a receiving DNA sequence (i.e., part of a coding or non-coding region of a gene) to transcriptionally or post-transcriptionally activate or suppress expression of the one or more target molecules. In some embodiments, the RNAi may be used as a therapeutic agent alone, or may be conjugated to one or more additional delivery, diagnostic or therapeutic agents.

In other embodiments, a universal nucleic acid binding molecule may be any suitable single-stranded antisense oligonucleotide which, when delivered to a target cell, associates with a may be used in antisense technology or therapy to bind a receiving nucleic acid sequence (e.g., DNA or RNA) to activate or inhibit (i.e., suppress or silence) the expression of one or more target molecules in the target cell via transcriptional or post-transcriptional activation or inhibition. Antisense oligonucleotides may be altered by one or more suitable modifications known in the art to confer persistence and protection against unwanted endogenous nucleases including, but not limited to, a phosphorothioate modification, an alkyl modification (e.g., addition of a methyl or methoxy-ethyl group), a gapmer modification, modifications to form locked nucleic acids (LNA), peptide nucleic acids (PNA), phosphoroamidate (PA) nucleic acid, or hexitol nucleic acids (HNA). In certain aspects, the oligonucleotide binds a segment of a receiving RNA sequence produced by a coding or non-coding region of a particular gene or DNA sequence to post-transcriptionally activate or suppress expression of one or more target molecules. The receiving RNA sequence may be a protein-coding RNA (e.g., mRNA) or a non-protein-coding RNA (e.g., IncRNA, tRNA, miRNA, rRNA, snRNA and others). In other aspects, the oligonucleotide binds a segment of a receiving DNA sequence (i.e., part of a coding or non-coding region of a gene to transcriptionally or post-transcriptionally activate or suppress expression of one or more target molecules. In some embodiments, the antisense oligonucleotide may be used as a therapeutic agent alone, conjugated to one or more additional delivery, diagnostic or therapeutic agents, or may be used to design an RNAi molecule, wherein the RNAi molecule may be used as a therapeutic agent.

In some embodiments, a universal nucleic acid binding molecule may be designed to modulate (i.e., activate or suppress/inhibit) any target molecule for therapeutic or research purposes. For example, the universal nucleic acid binding molecule may be used alone or conjugated to or otherwise administered with one or more additional therapeutic agents for treating a disease or condition as described in detail below. Moreover, the universal nucleic acid binding molecules may be used to target molecules that are part of a cellular regulation pathway in order to determine the effect that suppression or activation of the target molecule in relation to other molecules has on the pathway. In this sense, the universal nucleic acid binding molecules may be used in research methods for determining the mechanism of action in signaling pathways for drug discovery or for the discovery of other research tools used for in vivo or in vitro assays.

According to some embodiments, target molecules that may be modulated by the universal nucleic acid binding molecules described herein may include, but are not limited to, a wild type or variant molecule found in its native form, or may be a molecule that is prone to mutation by endogenous or exogenous factors, allelic diversity, and/or cross-species diversity including, but not limited to, wild-type or variant endogenous proteins that may or may not be associated with a disease, condition or desired physiological function or mechanism of action; exogenous proteins (i.e., viral proteins, bacterial proteins, fungal proteins, parasitic proteins, or any other microorganismal or environmental protein); proteins or genes associated with the development of cancer or resistance to cancer treatment (e.g., but not limited to, KRAS, NRAS, BRAF, ERK, STAT3, FLT3, ATM, BRCA1/2, EGFR, HER2, CEA, PSMA, VEGF, CA-125, CD19, CD20, p53, HRPT locus, NT5C2, INK4a/ARF); RNA molecules (e.g., coding RNA molecules (mRNA) or non-coding RNA molecules (miRNAs, tRNA, rRNA, and other non-coding RNA (ncRNA) molecules)); and DNA molecules and genetic variants or mutants of endogenous protein coding gene regions and/or regulatory gene regions (e.g., but not limited to, certain oncogenes that are mutated, overexpressed or underexpressed). In some embodiments, the universal nucleic acid binding molecules bind a viral RNA sequence via at least one non-Watson Crick or non-canonical paired base, thereby suppressing or activating the expression of a viral protein or an oncogene. In some embodiments, the universal nucleic acid binding molecules are designed to suppress or activate expression of a viral protein derived from a human immunodeficiency virus (HIV), an influenza virus (e.g., Influenzavirus A, Influenzavirus B, Influenzavirus C), hepatitis B virus (HBV), or hepatitis C virus (HCV). In some aspects, the universal nucleic acid binding molecules are designed to suppress or activate expression of an HIV protein (e.g., docking glycoprotein gp120, transmembrane glycoprotein gp41, p7, capsid protein p24, nucleocapsid protein p7, matrix protein p17, transcriptional transactivators p16 and p14, Vif protein p23, p27, rev protein p19) that is expressed from one or more HIV genes or gene regions (e.g., gag, pol, gag-pol, env, rev, nef, vif, vpr, vpu, tev).

In other embodiments, the universal nucleic acid binding molecules are designed to suppress or activate expression of a target gene or variant thereof which is associated with cancer or resistance to chemotherapy (or other cancer treatment) including, but not limited to, STAT3, KRAS, NRAS, BRAF, STAT3, FLT3, ATM, BRCA1/2, EGFR, p53, HRPT locus, NT5C2, or INK4a/ARF. The study of target genes that are associated with cancer or resistance to chemotherapy is ongoing in the field, but using the methods described here, a universal nucleic acid binding molecule may be developed for any known or future target gene.

In some embodiments, the universal nucleic acid binding molecules provided herein may include an inhibitory nucleic acid sequence or an activating nucleic acid sequence (e.g., an inhibitory RNA sequence, an inhibitory DNA sequence, an activating RNA sequence, or an activating DNA sequence) that binds a receiving nucleic acid sequence (e.g., a receiving RNA or DNA sequence) that may encode a target molecule. As described above, the target molecule may in certain embodiments be a protein, peptide, or polypeptide; or may itself be a noncoding RNA sequence. According to some embodiments, the inhibitory or activating RNA sequence may be designed using a method referred to herein as Multi-SubType Escape Reducing (MuSTER) design. In one embodiment, a MuSTER design method may include a step of generating an inhibitory or activating nucleic acid sequence by generating a consensus sequence derived from two or more receiving nucleic acid sequences of host or foreign origin (i.e., the consensus sequence may be derived from two or more viral species or strains, or from two or more animal species, or from two or more isoforms of the same target molecule), and converting the consensus sequence into an International Union of Pure and Applied Chemistry (IUPAC) sequence code by randomly or selectively replacing one or more U or G's with a corresponding ambiguous IUPAC coded base (Y or R) as described herein. In some embodiments, the consensus sequence may be derived from two or more nucleic acid sequences from two or more species of animals (e.g., sequences found in humans, rats, mice, rabbit, bovine, porcine, canine, etc.) or strains of a particular virus (e.g., sequences found in multiple strains of HIV, as described herein).

In an alternative embodiment, the MuSTER design method may include a step of generating an inhibitory or activating nucleic acid sequence by generating an artificial target sequence from one target nucleic acid sequence of host of foreign origin by randomly or selectively replacing one or more U or G's with a corresponding ambiguous IUPAC coded base (Y or R). shRNAs which do not pair with the target by only WCp may have the same or better activity than conventionally full WCp shRNAs. Thus, the alternative design method may be used to generate an si/shRNA against an artificial target sequence, i.e., a single natural target sequence, by replacing some U or G with an ambiguous base and then designing si/shRNAs against the resulting IUPAC code. In such case, the resulting guide strand will not pair WCp-only and may thus be a stronger and/or effective guide strand as compared to a guide strand that binds using WCp only.

The MuSTER design methods may also include a step of substituting each ambiguous base that allows for Watson Crick, non-Watson Crick, and non-canonical pairings. This step yields one or more candidate inhibitory or activating nucleic acid sequences. The resulting inhibitory or activating nucleic acid sequences using either of the steps described above may then be used as a single-stranded antisense oligonucleotide; or may be incorporated into an RNAi molecule, wherein the inhibitory or activating nucleic acid sequence acts as the guide strand (i.e., the antisense strand) of the RNAi molecule.

In some aspects, the receiving nucleic acid sequence (e.g., RNA or DNA) may be a consensus sequence that is generated from two or more selected receiving RNA sequences that encode two or more wild type or variant isotypes, escape variants, or mutants of the same protein, noncoding RNA or regulatory sequence. In some aspects, the two or more selected receiving RNA sequences are from a conserved sequence.

In certain embodiments, the selected receiving RNA sequences may be part of two or more viral genomes or nucleotide sequences (e.g., cDNA, or RNA) that are transcribed from the viral genome (i.e., nucleotide sequences that are a result of reverse transcription of an RNA virus or retrovirus or a result of transcription of the viral genome after integration with the host genome). In such embodiments, the MuSTER design method may include a step of generating an inhibitory or activating nucleic acid sequence by generating a consensus sequence from two or more clinical isolates. In this case, the two or more viral genomes may be derived from two or more clinical isolates. Clinical isolates are samples of a pathogen (e.g., virus, bacteria, or other microorganism) that are isolated from a biological sample of a subject infected with the pathogen, the sequences of which may be found on publicly available databases such as those found in Table 1 below. As such, in some embodiments, the two or more target RNA sequences that may be used to generate a consensus sequence for designing a universal nucleic acid binding molecule in accordance with the embodiments described herein may be derived from two or more sequences from clinical isolates found in one or more of the databases in Table 1 below.

TABLE 1
Viral sequence databases

| Virus | Database | Website |
|---|---|---|
| HIV-1 | Los Alamos HIV Database: | http://www.hiv.lanl.gov/content/sequence/HIV/mainpage.html |
| | Stanford HIV RT and | http://hivdb.stanford.edu |
| | Protease Sequence | |
| | Database: | |
| | NCBI/Genbank | http://www.ncbi.nlm.nih.gov/nuccore |
| HCV | Los Alamos HCV Database | http://hcv.lanl.gov/content/sequence/HCV/ToolsOutline.html |
| Influenza | NCBI Influenza Virus | http://www.ncbi.nlm.nih.gov/genomes/FLU/Database/nph-select.cgi?go=1 |
| | Resource (Bao, Bolotov | |
| | et al. 2008) | |

In some embodiments described above, the MuSTER method includes a step of converting the consensus sequence into an International Union of Pure and Applied Chemistry (IUPAC) sequence code. The IUPAC sequence includes a nucleotide code that includes several degenerate nucleotide code letters that represent one or more possible alternative bases that may be used at a particular position. Nucleotide code letters that represent more than one possible alternative bases are also known as “ambiguous bases.” Table 2 below shows the nucleotide code letters and corresponding base(s).

TABLE 2
IUPAC Sequence

| Nucleotide Code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . o r- | gap |

By converting the consensus sequence into an IUPAC sequence code, one can design a universal nucleic acid binding molecule that would bind one or more receiving nucleic acid sequences (e.g., RNA or DNA) that may differ by one or more nucleotides (i.e., the universal nucleic acid binding molecule may be used to bind one or more strains or variants of a particular target). The IUPAC sequence code is then transformed into a candidate universal nucleic acid binding molecule by substituting each ambiguous base with a base that allows for both Watson-Crick and non-Watson-Crick/non-canonical pairings: thymine (T) or uracil (U) is substituted with the IUPAC nucleotide code “R” to account for its Watson-Crick pairing base (A) or its non-Watson-Crick pairing base (G); and guanine (G) is substituted with the IUPAC nucleotide code “Y” to account for its Watson-Crick pairing base (C), its non-Watson-Crick pairing base (T) or (U), or its non-canonical base (G can pair with C, U, or G; U can pair with A or G; C can pair with C).

In some embodiments, the MuSTER methods may also include a step of selecting one or more inhibitory or activating nucleic acid molecules using a suitable selection method including, but not limited to, an RNAi selection method, an antisense oligonucleotide selection protocol, or an RNA-based transcriptional activation or inhibition selection method. In one aspect, the suitable selection method is an RNAi selection method such as an RNAi binding prediction algorithm. Suitable binding prediction algorithms are known in the art, and may include publicly and commercially available prediction tools including, but not limited to, DSir (http://biodev.extra.cea.fr/DSIR/DSIR.html) (Vert et al. 2006); siRNA at Whitehead (http://sirna.wi.mit.edu/); BLOCK-iT™ RNAi Designer at Life Technologies (http://RNAidesigner.invitrogen.com/RNAiexpress/); siDESIGN Center at Thermo Scientific (http://www.thermoscientificbio.com/design-center/); IDT SciTools RNAi Design at Integrated DNA Technologies (http://www.idtdna.com/scitools/applications/RNAi/RNAi.aspx); and German Cancer Research Center E-RNAi (http://www.dkfz.de/signaling/cgi-bin/e-RNAi3/settings.pl).

In other aspects, the suitable selection method may be an antisense oligonucleotide selection method or protocol. Antisense oligonucleotide selection methods may include the use of any tool or protocol which assesses and accounts for target accessibility, which is often related to the nucleic acid's secondary structure. (see https://www.idtdna.com/pages/docs/technical-reports/designing-antisense-oligonucleotides.pdf or http://sfold.wadsworth.org). Such methods may include algorithms and selection protocols which take into account characteristics of the target and antisense oligonucleotide sequences including, but not limited to, length of the antisense oligonucleotide, secondary and tertiary structures of the target sequence, protein binding sites on the target sequence, presence of CG motifs in the target sequence, potential for formation of tetraplexes within the antisense oligonucleotide, and known activity-increasing or decreasing motifs in the oligonucleotide (e.g., CCAC, TCCC, ACTC, GCCA, CTCT, GGGG, ACTG, TAA, CCGG, AAA), minimum free energy structure of the oligonucleotide.

In one embodiment, the MuSTER method may produce an antisense oligonucleotide molecule (also referred to as “MuSTER oligo,” “MuSTER oligonucleotide,” “MuSTER antisense oligo,” “MuSTER antisense oligonucleotide,” or the like) wherein the antisense oligonucleotide molecule is an inhibitory or activating nucleic acid sequence ((e.g., RNA, DNA, cDNA) that binds to a receiving nucleic acid molecule to suppress or activate the expression or activity of a target molecule such as a target nucleic acid sequence (e.g., RNA or DNA). In one embodiment, the antisense oligonucleotide molecule suppresses or activates the expression or activity of a target nucleic acid sequence (e.g., RNA or DNA) that is derived from a retrovirus, such as an HIV virus, an HCV virus, or an influenza virus. In such embodiments, the nucleic acid sequence (e.g., RNA or DNA) whose activity or expression is suppressed or activated by the inhibitory or activating nucleic acid sequence may be part of the retroviral genome or may be part of an mRNA sequence that is transcribed by the host after integration.

In another embodiment, the MuSTER method may produce an RNAi molecule (also referred to as “MuSTER RNAi,” “MuSTER designed RNAi,” or “MuSTER designed shRNA” or the like) that includes an inhibitory or activating nucleic acid sequence (e.g., RNA, DNA, cDNA) that binds to a receiving nucleic acid molecule to suppress or activate the expression or activity of a target molecule such as a target nucleic acid sequence (e.g., RNA or DNA). In one embodiment, the RNAi molecule suppresses or activates the expression or activity of a target pathologic nucleic acid sequence (e.g., RNA or DNA) that is derived from a retrovirus, such as an HIV virus, an HCV virus, or an influenza virus. In such embodiments, the nucleic acid sequence (e.g., RNA or DNA) whose activity or expression is suppressed or activated by the inhibitory or activating nucleic acid sequence may be part of the retroviral genome or may be part of an mRNA sequence that is transcribed by the host after integration.

In certain embodiments, the inhibitory or activating nucleic acid sequence may bind to at least a portion of a target viral RNA sequence that is derived from an HIV virus or a consensus sequence that is derived from a plurality of HIV viral strains, variants or mutants (i.e., a “target viral RNA sequence”). In such embodiments, the target viral RNA sequence may include an IUPAC sequence of URYCARUAYAUGGAYGAYYURUAUGURGG (SEQ ID NO:1) or YURGAYACRGGRGCAGAUGAUACAGUR (SEQ ID NO:2). In one aspect, expression of the target viral RNA sequence URYCARUAYAUGGAYGAYYURUAUGURGG (SEQ ID NO:1) is suppressed by an inhibitory RNA sequence selected from YARRTCRTCCATRTAYTGRYA (SEQ ID NO:3); TAYARRTCRTCCATRTAYTGR (SEQ ID NO:4); TAGGTCGTCCATGTATTGGTA (SEQ ID NO:5); or TATAGGTCGTCCATGTATTGG (SEQ ID NO:6). In another aspect, expression of the target viral RNA sequence YURGAYACRGGRGCAGAUGAUACAGUR (SEQ ID NO:2) is suppressed by an inhibitory RNA sequence selected from YACTGTATCATCTGCYCCYGT (SEQ ID NO:7); YADYACTGTATCATCTGCYCC (SEQ ID NO:8); DYACTGTATCATCTGCYCCYG (SEQ ID NO:9); ADYACTGTATCATCTGCYCCY (SEQ ID NO:10); TACTGTATCATCTGCTCCTGT (SEQ ID NO:11); TAGTACTGTATCATCTGCTCC (SEQ ID NO:12); GTACTGTATCATCTGCTCCTG (SEQ ID NO:13); or AGTACTGTATCATCTGCTCCT (SEQ ID NO:14).

The inhibitory or activating nucleic acid sequences may be used in or as an antisense oligonucleotide or a pre-processed siRNA molecule, or may be incorporated into an shRNA molecule which includes a passenger sequence and a guide sequence connected via a loop sequence. The passenger sequences, guide sequences (or a pre-processed siRNA sequence), or antisense oligonucleotide may be 18 nucleotides in length, 19 nucleotides in length, 20 nucleotides in length, 21 nucleotides in length, 22 nucleotides in length, 23 nucleotides in length, or any other suitable length. In the case of an RNAi molecule such as shRNA, the loop sequence may be between 4 and 25 nucleotides in length, or any other suitable length. In some embodiments, the inhibitory or activating nucleic acid sequence may be incorporated into an shRNA molecule which includes a sequence selected from the following sequences in Table 3 below (passenger sequence is underlined, guide sequence is in bold, loop sequence is in italics):

TABLE 3
MuSTER designed shRNA sequences

| shRNA | shRNA encoding DNA | Target viral RNA |
|---|---|---|
| ID | template (5′-3′) | sequence (5′-3′) |
| #3-2: | agcgATACCAATACATGGACGACCTA | URYCARUAYAUGGAYGAYYUR |
| | tagtgaagccacagatgtaTAGGTCGTCCA | UAUGURGG (SEQ ID NO: 1) |
| | TGTATTGGTAgtgcc (SEQ ID NO: 15) | |
| #3-3 | agcgGCCAATACATGGACGACCTATA | URYCARUAYAUGGAYGAYYUR |
| | tagtgaagccacagatgtaTATAGGTCGTC | UAUGURGG (SEQ ID NO: 1) |
| | CATGTATTGGttgcc (SEQ ID NO: 16) | |
| #4-1 | agcgAACAGGAGCAGATGATACAGT | YURGAYACRGGRGCAGAUGAU |
| | AtagtgaagccacagatgtaTACTGTATCAT | ACAGUR (SEQ ID NO: 2) |
| | CTGCTCCTGTgtgcc (SEQ ID NO: 17) | |
| #4-2 | agcgGGGAGCAGATGATACAGTACT | YURGAYACRGGRGCAGAUGAU |
| | AtagtgaagccacagatgtaTAGTACTGTAT | ACAGUR (SEQ ID NO: 2) |
| | CATCTGCTCCttgcc (SEQ ID NO: 18) | |
| #4-3 | agcgGCAGGAGCAGATGATACAGTA | YURGAYACRGGRGCAGAUGAU |
| | CtagtgaagccacagatgtaGTACTGTATC | ACAGUR (SEQ ID NO: 2) |
| | ATCTGCTCCTGttgcc (SEQ ID NO: 19) | |
| #4-4 | agcgAAGGAGCAGATGATACAGTACT | YURGAYACRGGRGCAGAUGAU |
| | tagtgaagccacagatgtaAGTACTGTATC | ACAGUR (SEQ ID NO: 2) |
| | ATCTGCTCCTgtgcc (SEQ ID NO: 20) | |

In other embodiment, the inhibitory or activating nucleic acid sequence may be an antisense oligonucleotide selected from the following sequences shown below:

Antisense oligonucleotide (5′-3′)
(SEQ ID NO: 5)
TAGGTCGTCCATGTATTGGTA
(SEQ ID NO: 6)
TATAGGTCGTCCATGTATTGG
(SEQ ID NO: 11)
TACTGTATCATCTGCTCCTGT
(SEQ ID NO: 12)
TAGTACTGTATCATCTGCTCC
(SEQ ID NO: 13)
GTACTGTATCATCTGCTCCTG
(SEQ ID NO: 14)
AGTACTGTATCATCTGCTCCT

A lot of effort has been spent in the last decade to find anti-HIV nucleic acid binding molecules such as antisense oligonucleotides, shRNAs, and siRNAs—for example—for gene therapy as RNAs are not prone to immune response while proteins are. The practical utility is that previous oligonucleotides and shRNAs/siRNAs targeting pathogens were rendered ineffective by naturally occurring mutants hampering the applicability in therapy. By designing universal sh/siRNAs that can tolerate naturally occurring mutations, the problem of mutant escape from such nucleic acid binding molecules may be solved.

Since no cure against HIV has yet been discovered, but gene therapy is a very promising option, finding escape-proof sequences is highly desired. The same holds true for infections with HCV or HBV for example against which no effective cure exists while the incidence is on the rise; as well as for a treatment against chemotherapy resistance development through the mutation of targeted host genes.

### Pharmaceutical Compositions

According to some embodiments, the universal nucleic acid binding molecules described herein may be part of a pharmaceutical composition. Such a pharmaceutical composition may include one or more universal nucleic acid binding molecules and a pharmaceutically acceptable carrier. In some embodiments, the pharmaceutical composition may include a single universal nucleic acid binding molecule that targets and binds a first receiving nucleic acid sequence (e.g., RNA or DNA), or alternatively, may include one or more additional universal nucleic acid binding molecules that target and bind a second receiving nucleic acid sequence (e.g., RNA or DNA), a third receiving nucleic acid sequence (e.g., RNA or DNA), or any number of additional receiving nucleic acid sequences.

A “pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. Such a carrier may comprise, for example, a liquid, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, or some combination thereof.

Each component of the carrier is “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) natural polymers such as gelatin, collagen, fibrin, fibrinogen, laminin, decorin, hyaluronan, alginate and chitosan; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as trimethylene carbonate, ethyl oleate and ethyl laurate; (13) agar; (14) buffering agents, such as magnesium hydroxide and aluminum hydroxide; (15) alginic acid (or alginate); (16) pyrogen-free water; (17) isotonic saline; (18) Ringer's solution; (19) alcohol, such as ethyl alcohol and propane alcohol; (20) phosphate buffer solutions; (21) thermoplastics, such as polylactic acid, polyglycolic acid, (22) polyesters, such as polycaprolactone; (23) self-assembling peptides; and (24) other non-toxic compatible substances employed in pharmaceutical formulations such as acetone.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

In one embodiment, the pharmaceutically acceptable carrier is an aqueous carrier, e.g. buffered saline and the like. In certain embodiments, the pharmaceutically acceptable carrier is a polar solvent, e.g. acetone and alcohol.

The concentration of universal nucleic acid binding molecules in these formulations can vary widely, and will be selected primarily based on fluid volumes, viscosities, organ size, body weight and the like in accordance with the particular mode of administration selected and the biological system's needs.

### Methods of Treatment

In some embodiments, methods of treating a subject that is suffering from a disease or condition that is associated with one or more target molecules that are prone to mutation or allelic diversity. Target molecules that may be associated with a disease or condition may include proteins, peptides, polypeptides, noncoding nucleic acid sequences, coding nucleic acid sequences, or other nucleotide sequences. In one embodiment, the methods are directed to treatment of a subject that is infected with a target virus. In another embodiment, the methods are directed to treatment of a subject that has a chemotherapy-resistant cancer. Such methods include administering a therapeutically effective amount of a universal nucleic acid binding molecule or pharmaceutically composition thereof to the subject. According to some embodiments, the universal nucleic acid binding molecule includes an inhibitory or activating nucleic acid sequence which binds a target viral RNA sequence or variant thereof via at least one non-Watson-Crick or non-canonical paired base, such as those described in accordance with the embodiments described herein. The treatment may be used to treat any suitable viral infection including, but not limited to, human immunodeficiency virus (HIV), an influenza virus (e.g., Influenzavirus A, Influenzavirus B, Influenzavirus C), hepatitis B virus (HBV), or hepatitis C virus (HCV). In some embodiments, the treatment may be used to treat any strain or mutant version of such viral infection.

According to other embodiments, the universal nucleic acid binding molecule includes an inhibitory or activating RNA sequence which binds a target oncogene or variant thereof that is associated with resistance to chemotherapy treatment via at least one non-Watson-Crick or non-canonical paired base, such as those described in accordance with the embodiments described herein. The treatment may be used to treat any suitable chemotherapy-resistant cancer, or cancers which have secondary chemotherapy resistance (i.e., cancers which are associated with mutations that confer resistance after starting treatment).

The treatment may include administration of one universal nucleic acid binding molecule or pharmaceutical composition thereof which binds one target viral RNA sequence or oncogene, or may include administration of two or more universal nucleic acid binding molecules or a pharmaceutical composition thereof, wherein each universal nucleic acid binding molecule targets a different target viral RNA sequence or oncogene. This combination or combinatory treatment may be administered to increase the effectiveness of the treatment.

The terms “treat,” “treating,” or “treatment” as used herein with regards to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. For example, a treatment with a universal nucleic acid binding molecule or pharmaceutical composition thereof may be used to treat an active viral infection in a subject by suppressing the expression of one or more viral proteins that are involved with viral replication, integration or assembly, thereby preventing the spread of the virus; or a cancer which may or may not be resistant to chemotherapy. The treatments described herein may be used in any suitable subject, including a human subject or any mammalian or avian subject that needs treatment in accordance with the methods described herein (e.g., dogs, cats, horses, rabbits, mice, rats, pigs, cows).

A universal nucleic acid binding molecule or pharmaceutical composition thereof can be administered to a biological system by any administration route known in the art, including without limitation, oral, enteral, buccal, nasal, topical, rectal, vaginal, aerosol, transmucosal, epidermal, transdermal, dermal, ophthalmic, pulmonary, subcutaneous, and/or parenteral administration. The pharmaceutical compositions can be administered in a variety of unit dosage forms depending upon the method of administration. In one embodiment, the universal nucleic acid binding molecule or pharmaceutical composition thereof is administered parenterally. A parenteral administration refers to an administration route that typically relates to injection which includes but is not limited to intravenous, intramuscular, intraarterial, intrathecal, intracapsular, intraorbital, intra cardiac, intradermal, intraperitoneal, transtracheal, subcutaneous, subcuticular, intraarticular, subcapsular, subarachnoid, intraspinal, and/or intrasternal injection and/or infusion.

In some embodiments, the universal nucleic acid binding molecules may be administered with a pharmaceutically effective carrier that allows the universal nucleic acid binding molecules to be delivered locally or systemically to one or more target cells (i.e., virally infected cells or cancer cells) or target organs by one or more suitable universal nucleic acid binding delivery methods known in the art including, but not limited to, viral delivery, liposomal delivery, nanoparticle delivery, targeted delivery (e.g., using an antibody, aptamer or other targeting molecule to facilitate delivery), direct administration into target organs, systemic injection of naked universal nucleic acid binding molecules, and eukaryotic transcription plasmid delivery to produce shRNA inside of the target cells.

A universal nucleic acid binding molecule or a pharmaceutical composition thereof can be given to a subject in the form of formulations or preparations suitable for each administration route. The formulations useful in the methods of the invention include one or more universal nucleic acid binding molecules, one or more pharmaceutically acceptable carriers therefor, and optionally other therapeutic ingredients. The formulations may conveniently be presented in unit dosage form and may be prepared by any methods well known in the art of pharmacy. The amount of active ingredient which can be combined with a carrier material to produce a single dosage form will vary depending upon the subject being treated and the particular mode of administration. The amount of a universal nucleic acid binding molecule which can be combined with a carrier material to produce a pharmaceutically effective dose will generally be that amount of a universal nucleic acid binding molecule which produces a therapeutic effect.

Methods of preparing these formulations or compositions include the step of bringing into association a universal nucleic acid binding molecule with one or more pharmaceutically acceptable carriers and, optionally, one or more accessory ingredients. In general, the formulations are prepared by uniformly and intimately bringing into association a universal nucleic acid binding molecule with liquid carriers, or finely divided solid carriers, or both.

Formulations suitable for parenteral administration comprise a universal nucleic acid binding molecule in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e.g., such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents, viscous agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In one embodiment of the invention, a universal nucleic acid binding molecule or pharmaceutical composition thereof is delivered to a disease or infection site in a therapeutically effective dose. A “therapeutically effective amount” or a “therapeutically effective dose” is an amount of a universal nucleic acid binding molecule that produces a desired therapeutic effect in a subject, such as preventing or treating a target condition or alleviating symptoms associated with the condition. The most effective results in terms of efficacy of treatment in a given subject will vary depending upon a variety of factors, including but not limited to the characteristics of the universal nucleic acid binding molecule, the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

As described above, regular si/shRNAs (small interfering/small hairpin RNAs) are typically designed by calculating active sequences based on a naturally existing target sequence in the standard 4-letter code. In case of mutating target like HIV, subtypes or sub-strains are analyzed for conserved regions, then the majority sequence inside these chosen and the si/shRNA designed. The resulting sequence pool is small and decreases with the mutagenicity of the target organism. Once the target mutates these conventional RNAs lose activity due to bulge formation/mismatch creation (Joseph and Osman 2012). This plays a major role for loss of efficacy in the treatment of infectious diseases like HIV or other pathogens which are prone to mutation and thus therapeutic “escape.” Furthermore infectious agents often exist in subtypes or sub-strains which already have a more or less distinctive genetic setup at infection time point. A general therapeutic universal nucleic acid binding molecule approach thus needs to be able to cope with distinctive RNA sequences at onset of, as well as developing sequences during the infection. As described in the examples below, the approach to the design of regulatory RNAs is, therefore, not based on a naturally occurring sequence. Instead, an artificial sequence is created from as many subtypes or sub-strains as possible found worldwide. That information is then encoded in the IUPAC code which covers mutation information by addition of several more than the typical 4 base letters. The artificial sequence is then analyzed for their composition in terms of conserved sequences covering non-Watson-Crick/non-canonical pairing. This yields a sequence selection which would otherwise be excluded from conventional antisense or si/shRNA design. The IUPAC sequence is then converted into an artificial template strain that has the IUPAC encoded Y (U or C possible) and R (G or A possible) positions replaced with the corresponding non-Watson-Crick and non-canonical pairing (G can pair with C, U, or G; U can pair with A or G; C can pair with C). Based on that artificial sequence the si/shRNAs are selected. Resulting predictions are reviewed for Non-Watson-Crick/non-canonical positions in the seed region (disfavoring of non-Watson-Crick/non-canonical in 2.-8. nucleotide from the 5′ end of the guide strand to avoid off target pairing and positions 10 & 11 to avoid slicing blockage) and frequency (avoid non-Watson-Crick/non-canonical pairings next to each other and more than 4 in one sequence). These antisense oligonucleotides and/or si/shRNAs are referred to as MuSTER antisense oligonucleotides and/or si/shRNAs (Multi-SubType Escape Reducing) or MuSTER designed antisense oligonucleotides and/or si/shRNAs. The results in dual luciferase assays (HEK 293T), an HIV HXB2 integrated infection deficient pro-virus packaging (HeLa cells), replication competent NL4-3 packaging assay (HEK 293T cells) and a long term virus propagation assay with HIV IIIB (CEM CCR5 T cell line) demonstrate a high activity and improved effectivity of the MuSTER design as compared to conventional designs.

### Example 1

### Design of Constructs

Since non-Watson Crick/non-canonical pairs allow a single si/shRNA to cover several variants of target mRNA it could be used to generate mutation resistant RNAi. This would allow overcoming the current limitation of the RNAi method in therapy. It is noted that although the design methods described below are used to design RNAi molecules, one skilled in the art would understand that the novel design methods for designing universal nucleic acid binding molecules may also be applied to antisense technology and transcriptional activation or silencing by si/shRNAs as described above.

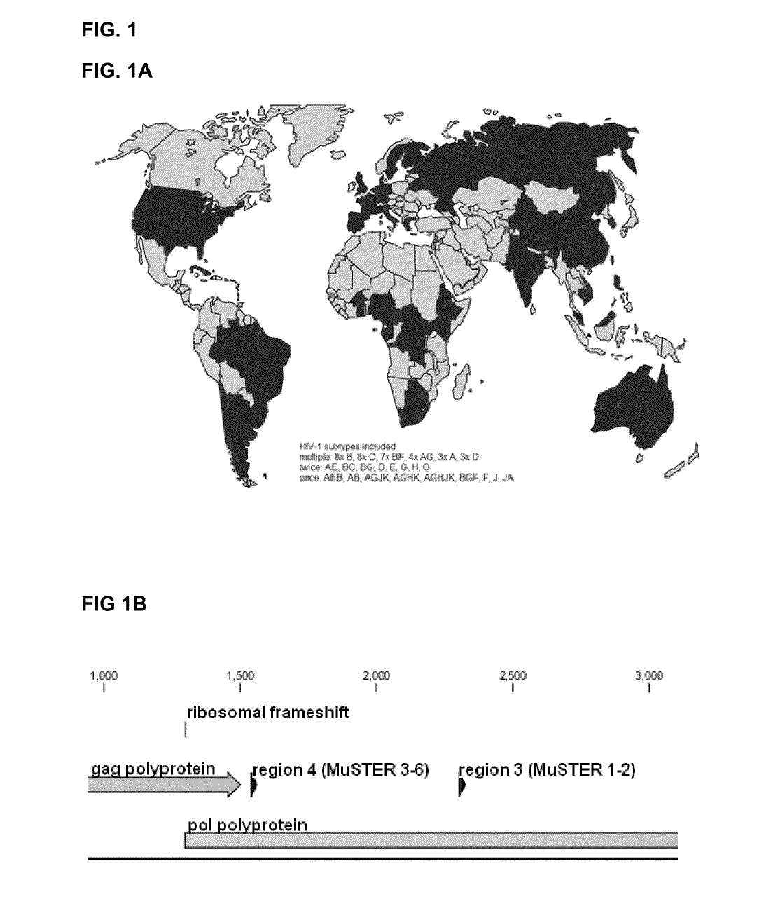

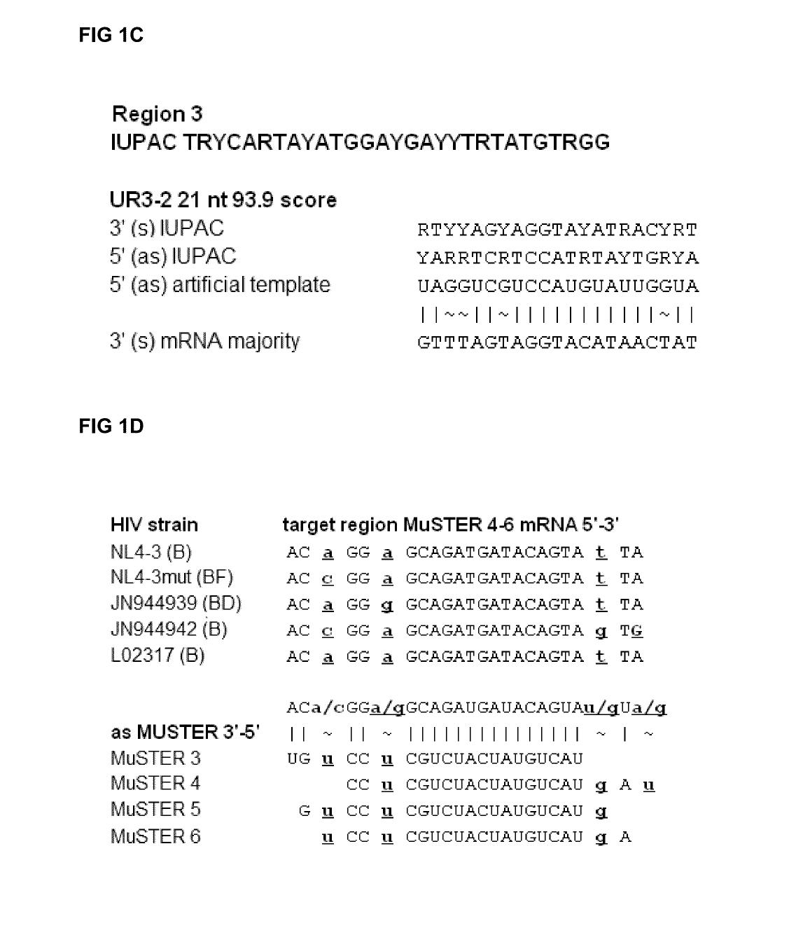

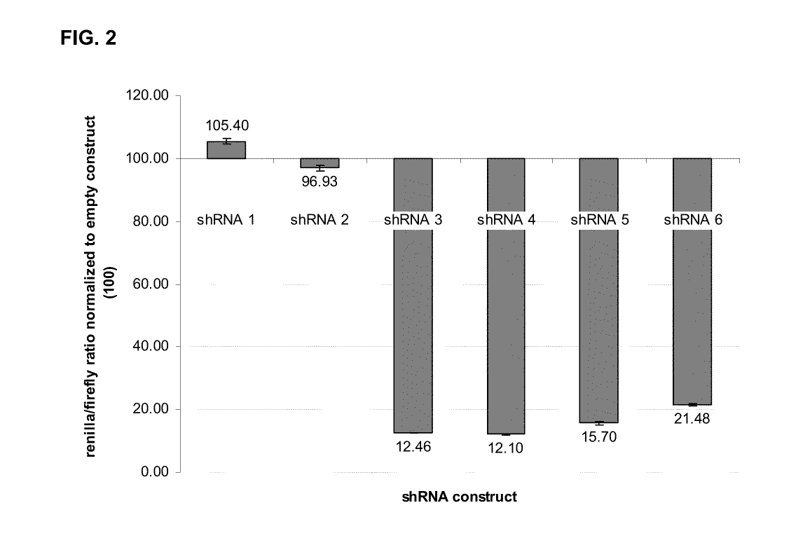

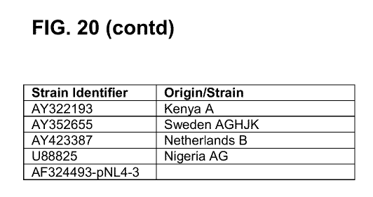

To test this, an artificial consensus sequence of 54 different clinical isolates found worldwide was generated for HIV (FIG. 1A), the sequences available at NCBI (see FIG. 20). This artificial sequence was encoded in IUPAC code, which contains a letter for ambiguous bases depending on the mutations occurring at each position. As next step every stretch was extracted that contained only the letters for bases Adenine, Guanine, Cytosine and Uracil (A, G, C, U) and the ambiguous letters for Cytosine or Uracil (pairing with Guanine, letter Y) and Guanine or Adenine (pairing with Uracil, letter R). These stretches would be conserved while targeted by fully complementary (including non-Watson Crick/non-canonical pairing) si/shRNAs. For all of these naturally occurring HIV subtypes no conventional si/shRNA was possible to design due to too high mutation rate of the virus. But several stretches of which were at least 22 nucleotides length the could be covered by MuSTER si/shRNAs were found and the resulting potential target sites are shown in Table 4 below.

TABLE 4
Target sequences (IUPAC)

| | Length | Region of | SEQ ID |
|---|---|---|---|
| Target Sequence | (nt) | gag-pol | NO |
| ARRUGGAGRAARYURGURGAYUUYAGRGARYU | 32 | region 1 | 21 |
| URYCARUAYAUGGAYGAYYURUAUGURGG | 29 | region 3 | 1 |
| RAUGAYAUCACARAARYURGURGGRAAR | 28 | region 2 | 22 |
| YURGAYACRGGRGCAGAUGAUACAGUR | 27 | region 4 | 2 |
| AARARRGGYUGYURGAARUGUR | 22 | region 5 | 23 |
| RRGGGARRRAGAURRGUGCRAG | 22 | region 6 | 24 |

Each of these sequences was transformed into an artificial DNA sequence where the ambiguous bases were replaced by a base that allowed both Watson-Crick and non-Watson Crick/non-canonical pairings (Thymine, the DNA analogue of Uracil for letter R and Guanine for letter Y). These artificial sequences were analyzed by the shRNA prediction tool DSir (available online) for effective si/shRNAs. As parameter, a length of 21 nucleotides (the longest prediction possible with DSir) with siRNA prediction was chosen. Predictions with highly ranked scores (above score of 75) were selected and analyzed for the position of the non-Watson Crick pairing bases. Every prediction which had a wobble (non-Watson-Crick pairing) at position 8-12 from the 3′ end of the guide strand was excluded as previous reports indicated these would interfere with the siRNA activity.

The remaining 6 sequences predicted to be effective si/shRNAs were extended by one nucleotide on the 5′ end of the guide strand (full complementarity to the target strand) and the resulting 22 nucleotide long sequences placed into human pre-microRNA 30 based backbones as described in (Du, Yonekubo et al. 2006). The resulting designs are shown in Table 3 above.

These would be processed into sequences that align as follows (“3′ IUPAC” represents the complementary sequence to the target RNA sequence; “5′ as RNA represents the guide sequence of the shRNA or the inhibitory (or activating) RNA or DNA sequence; and “3′ mRNA majority” represents the mRNA target viral RNA sequence):

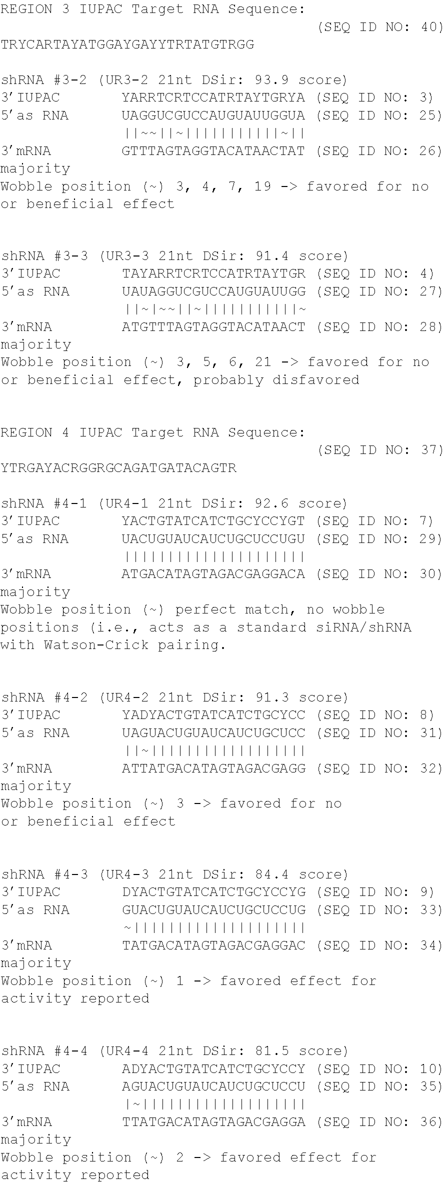

### Example 2

### Activity of shRNA Constructs

Materials and Methods

Construct Generation, Plasmids and HIV Strains.

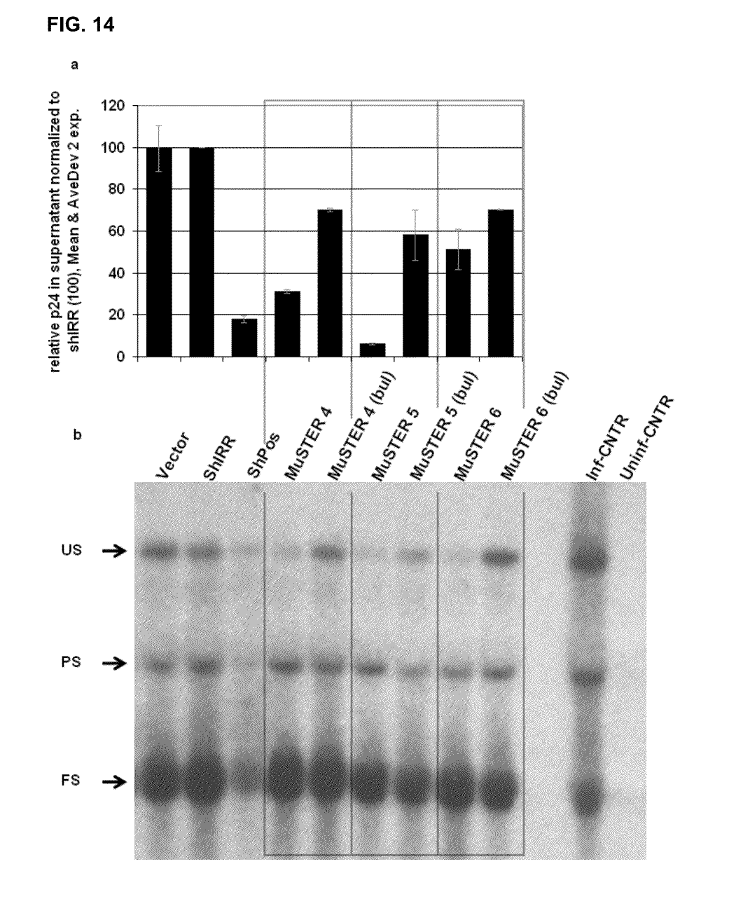

From the HIV strain HXB2 fb a part of gag-pol coding region was cloned into psiCheck2.1 3′ UTR with the primers gag-pol fw 5′ atatCTCGAGCTTCAGAGCAGACCAGAGCC (SEQ ID NO:38) and gag-pol rev 5′ tataGCGGCCGCTCCCCACCTCAACAGATGTT (SEQ ID NO:39) with restriction sites for XhoI and NotI restriction sites, in order to generate the target plasmid for the luciferase assays (psiR42). Used HIV-1 provirus plasmids were subtype B NL4-3 (#AF324493 Genbank), subtype B AIDS Repository pSG3.1 (#L02317 Genbank), subtype BD AIDS Repository pCH040.c (#JN944939 Genbank), subtype B AIDS Repository pCH106.c (#JN944942 Genbank). pNL4-3(mut) was generated by PCR amplification of a fragment of the gag-pol region of 1978 nucleotide length from BF subtype AF005495 to generate a gag-pol mutant strain of similar expression rate as NL-4-3. Primers used were BFfw 5′ GGAAGTGATATAGCTGGAACTACTAGTACC 3′ (SEQ ID NO:41) and BFrev 5′ atatatTCCGGATCTTTTAGAATCTCCCTGTTTTCTG 3′ (SEQ ID NO:42) and the fragment digested with SpeI and BspEI prior cloning into pNL4-3. The shRNA plasmids were generated by digesting pHIV7 (Li et al. 2003) with BamHI and DraII and inserting a multiple cloning site with primers HIVreinsert fw 5′ CGCGACTCTAGATCATGGATCCTCCGGACTGCACTCTAGATAGGTCACCACCGTC GACTAGCCGTACCT 3′ (SEQ ID NO:43) and HIV reinsert 5′ ACTATAGGGCGAATTGGGTACC (SEQ ID NO:44). The resulting plasmid pHIV-empty was digested with Kpn2I and Eco91I and a PCR fragment with the CMV-intron(premiR30)-GFP-partial polyA cassette from pSM30-GFP (Du et al. 2006, which is hereby incorporated by reference as if fully set forth herein) generated by primers HIV8miR30fw 5′ TATTCGCACTGGATACGATCCGGATGATTCTGTGGATAACCGT (SEQ ID NO:45) and HIV8miR30 rev 5′ AATATCCTCCTTAGTTCCGGTGACCTAGAATGCAGTGAAAAAAATG (SEQ ID NO:46) and ligated into the backbone to generate pHIV8. The individual shRNA plasmids were cloned by digestion of pHIV8 with Esp3I and ligation with the annealed and phosphorylated oligonucleotides depicted in FIG. 13.

Dual Luciferase Assay.

HEK 293T (ATCC) cells were seeded at a density of 300.000 cells per well in 24 well plates and, 24 hours later, 300 nanograms (ng) of the target gag-pol psiCheck plasmid psiR43 were co-transfected with 500 ng of the shRNA expressing plasmid or the empty construct pHIV8 using Lipofectamine 2000 following the manufacturer's protocol. Cells were harvested 48 hours post transfection and dual luciferase assays (Promega) were performed.

p24 Assay.

100.000 HlfB (AIDS repository) or HEK 293T cells (ATCC) were seeded one day in advance in 24 wells plates. HEK293T (ATCC) were transfected transiently with 200 ng of pNL4-3 (AIDS repository) and 338 ng of shRNA expressing or empty construct with Lipofectamine 2000 according to manufacturer's protocol and p24 levels were measured 48 hrs later. Total DNA had been adjusted to 800 ng with empty construct (pHIV8)

To assess dose effects HlfB with genomically integrated provirus (but rev/env deficiency mutation) were co-transfected with 338 ng or 26 ng of shRNA constructs as well as 100 ng rev (Li et al. 2003) and gp120 (kindly provided by Dr. Burnett, City of Hope) encoding plasmid with Lipofectamine 2000 according to manufacturer's protocol. Total DNA had been adjusted to 800 ng with empty construct (pHIV8). Forty eight hours post transfection supernatant was harvested, spun down at room temperature for 5 minutes at 300 rpm to remove cell debris, and p24 was measured with the Alliance HIV-1 p24 antigen ELISA kit (Perkin Elmer, NEK050001KT) according to manufacturer's protocol in a Victor 3 1420 (Perkin Elmer) at 450 nm & 490 nm.

For combined p24 analysis and Northern Blot HEK 293T cells were seeded into 6 well multiwall plates in 2 ml media and 24 hours later 1.7 μg RNAi plasmid and 1 μg pNL4-3 into HEK 293T cells with Lipofectamine 2000 cotransfected (total DNA was adjusted with empty vector to 4 μg) according to manufacturer's protocols. After 48 hours supernatant was collected.

Northern Blot.

Forty-eight hours post co-transfection as described in “p24 assay”, cells were pelleted by centrifugation Cell pellets were resuspended in 1 ml of RNA-STAT60 (TEL-TEST B Inc., Friendswood, Tex., USA) and RNA was extracted following manufacturer instructions. Fifteen micrograms of RNA from each sample were analyzed on a 0.8% Agarose denaturing gel. To prepare the gel, 0.8 g RNase free Agarose were added to 74 ml DEPC H₂O and microwave to dissolve. Following incubation at 55° C. for 15 minutes to equilibrate, 10 ml of 10×MOPS pH7.0 and 16 ml of 37% Formaldehyde were added to the Agarose solution in a ventilated hood and the gel was poured and allowed to set for 1 hour at RT.

To visualize the integrity of the RNA samples, EtBr at a final concentration of 10 μg/ml was directly added to the samples Formaldehyde gel-loading dye. The gel was run in 1×MOPS buffer for about 3 hours at ˜5V/cm and at the end of the run it was briefly rinsed and then soaked for 10 min in 10×SSC transfer buffer. The RNA was transferred from the agarose gel to a positively charged nylon membrane (Amersham Hybond N+) by a downward capillary transfer overnight. The next day the membrane was rinsed in 2×SSC buffer, UV cross-linked, and pre-hybridized in 10 ml of ULTRAhyb hybridization solution (Ambion) for 1 hour at 40° C. For the splicing analysis forty picomoles of a γ-³²P labeled oligo probe complementary to a region located within the HIV-Nef gene were directly added to the pre-hybridization solution and the hybridization reaction was carried out over night at 40° C. The next day, the membrane was washed at 42° C. twice for 15 minutes each with 2×SSC, 0.1% SDS and two more times (15 min. each) with 0.1×SSC, 0.1% SDS. To visualize the unspliced and spliced forms of HIV RNA, the filter was exposed to film at −80° C. for 4.5 hours. For detection of the processed shRNAs, twenty microgram of total RNA were fractionated in 7 M-8% PAGE, and transferred onto a Hybond-N+ membrane (Amersham Pharmacia Biotech). Twenty picomoles of γ-³²P-radiolabeled 21-mer probes complementary to each respective siRNA-antisense sequences were used for the hybridization reactions. Hybridizations were performed sequentially with washes in between each set. The mature form of shRNAS1 was detected first. After removal of the probe and subsequent washings the probes fully complementary to the bulged control constructs (see FIG. 13, MuSTER 4B, 5B, and 6B) were added and finally, following the removal of these probes, the detection of the mature active shRNA (see FIG. 13; MuSTER 4, 5, and 6) was performed by adding the corresponding complementary labeled probes. All hybridization reactions were carried out over night at 37° C. Each wash was performed 24 hours after the hybridization reaction at 40° C. twice for 15 minutes each with 2×SSC, 0.1% SDS and two more times (15 min. each) with 0.1×SSC, 0.1% SDS. The filter was exposed to film at −80° C. over night to detect S1 and MuSTER4, 5, 6, and 36 hours to detect MuSTER 4B, 5B, 6B. The detection is qualitative and not quantitative since Northern probes, designed to be complementary to the bulged controls, have the ability to cross-hybridize with the corresponding active siRNAs. Some of the probes can also self-pair reducing the detection of the matching siRNA. All probes were heated to 90° C. for 3 minutes prior adding them to the pre-hybridization solution. Probe sequences are shown below:

Nef-Rev :
(SEQ ID NO: 47)
5′-GCCATCGATATTGTTAGCTGCTGTATTGCTACTTGTG
s1 shRNA:
(SEQ ID NO: 48)
5′-GCGGAGACAGCGACGAAGAGC
U6 snRN:A
(SEQ ID NO: 49)
5′-TATGGAACGCTTCACGAATTTG
MuSTER 4:
(SEQ ID NO: 50)
5′-AGGAGCAGATGATACAGTACTA
MuSTER 4B:
(SEQ ID NO: 51)
5′-AGGAGCAGTAACATACAGTACTA
MuSTER 5:
(SEQ ID NO: 52)
5′-ACAGGAGCAGATGATACAGTAC
MuSTER 5B:
(SEQ ID NO: 53)
5′-ACAGGAGCTCCTTGATACAGTAC
MuSTER 6:
(SEQ ID NO: 54)
5′-CAGGAGCAGATGATACAGTACT
MuSTER 6B:
(SEQ ID NO: 55)
5′-CAGGAGCACTTAGATACAGTACT

Contrary to conventional designs MuSTER rules define several target regions in multi subtype/clade HIV-1 alignments as conserved.

To overcome current limitations of RNAi therapy for HIV due to viral escape, the inclusion of base pairs besides conventional WCp was included in the design of shRNAs to cover several variants of target RNA. In other words this new class of si/shRNA should, at least to some degree, achieve mutation resistant RNAi.

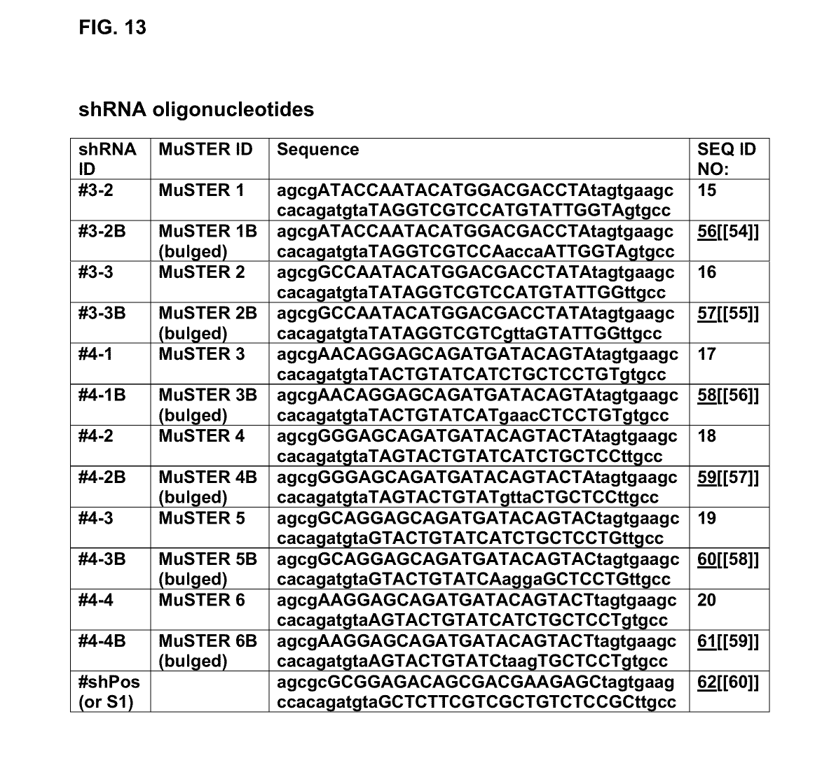

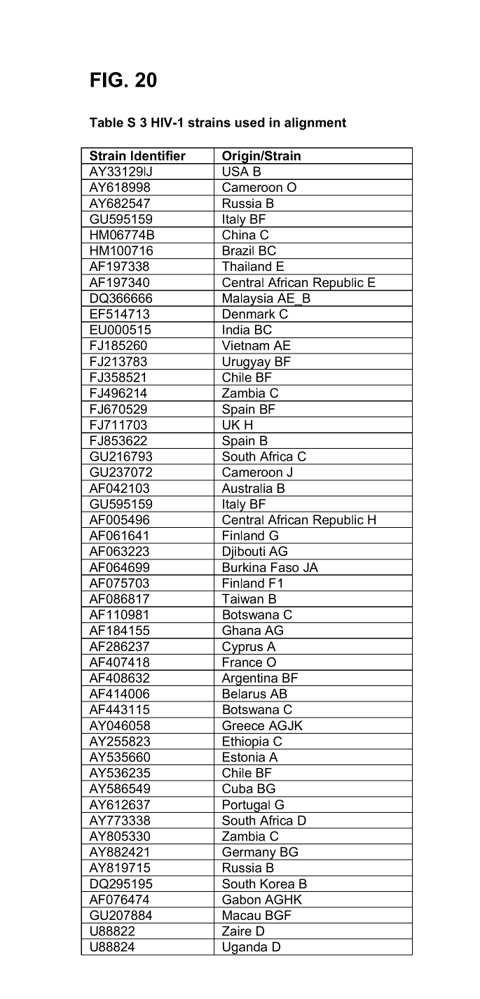

A consensus HIV sequence based on 54 different clinical isolates found globally (FIG. 1A, the sequences are available at NCBI with the identifiers shown in FIG. 20) was generated as a starting point. These naturally occurring subtypes and variants resulting from the high mutation rate of the virus could not be simultaneously and effectively targeted by a canonical si/shRNA due to exclusively using Watson/Crick pairing. However RNAi was possible with pairings of G-U as well as the non-canonical pairings of C-U and G-G as these are unlikely to interfere with A-form helical formation and thus should support an active RISC. This is because CGG repeats found in the mRNA associated with human fragile X-associated tremor ataxia syndrome still fold into an A-form helical structures (FIG. 12A depicts the predicted hydrogen bonds between the base pairs and FIG. 12B visualizes the A-form helix with GG pairings from structure “3r1c 1 AS” (see Kiliszek et al.) in Pymol 1.5.0.4 to demonstrate the A-form helix of GG pairings in naturally occurring RNA double strand formation). The consensus was encoded according to IUPAC conventions which allow precise definition of ambiguous positions. All target stretches that contained 100% conserved bases (A, G, C, U) as well as the ambiguous positions that would allow pairing of Cytosine or Uracil with Guanine (Y) and Guanine or Adenine with Uracil (R) were extrapolated. While no stretches could be found that would be conserved according to conventional si/shRNA design rules, several regions would be deemed conserved according to the MuSTER rules set forth herein (See FIG. 19 for the python script used for this filtering, according to one embodiment). These could be targeted by si/shRNAs containing canonical and/or non-canonical base-pairings (nWCp). Each of these sequence stretches was converted into a DNA target sequence where each base would allow Watson/Crick, non-Watson/Crick and non-canonical pairings in the antisense strand (Uracil to target IUPAC R and Guanine to target IUPAC Y). Thus the resulting target DNA strand is artificial and very likely never found in nature in this composition, but it covers the full natural mutational pattern. These artificial target sequences were used to predict 21 nucleotide long effective siRNAs with DSir algorithm (see http://biodev.extra.cea.fr/DSIR/; Vert et al. 2006, which is hereby incorporated by reference as if fully set forth herein) and predictions with scores above 75 were selected for further analyses. Based on the location and proportion of guide strand positions without Watson/Crick pairings from six possible target regions two in the HIV gag-pol gene were selected. (FIG. 13 and FIGS. 1C and 1D). For these regions six RNAi trigger capable of binding multiple viral subtypes were predicted to be highly active (referred to as MuSTER—Multi-SubType, Escape Reducing). The antisense sequences were extended by one nucleotide on the 5′ end of the guide strand (following MuSTER complementary definition with the target strand) and the resulting 22 nucleotide long sequences were inserted into a human pre-microRNA 30 scaffold (Du et al. 2006, which is hereby incorporated by reference as if fully set forth herein) to generate expression cassettes.

Region 4 Constructs Provide High Knockdown Activity Against a Luciferase Reporter Construct with NL4-3 Based Target Region

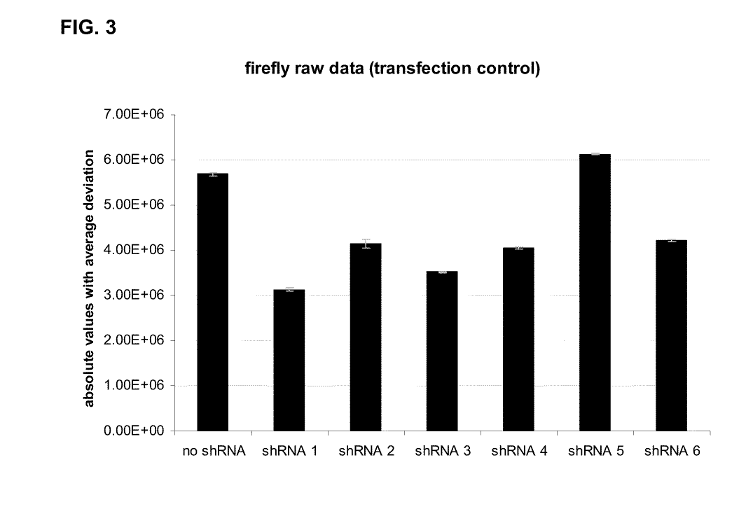

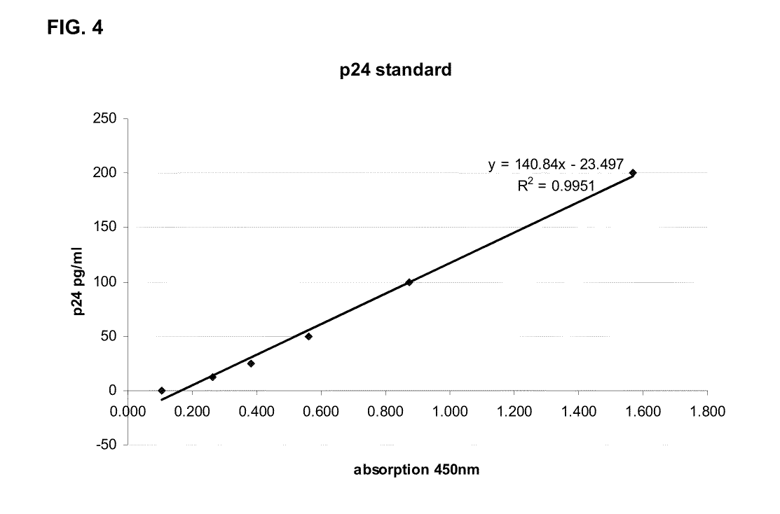

. First, a dual luciferase assay was used to test the efficacy of the MuSTER shRNAs against the targeted HIV region 3 and 4. A psiCheck reporter system containing 1056 nucleotides of the gag-pol region within its 3′UTR was co-transfected with the MuSTER shRNAs-expressing constructs in HEK293T cells. While region 3 targeting shRNAs 3-2 (MuSTER 1) and 3-3 (MuSTER 2) (FIG. 2, shRNA 1 & shRNA 2) did not lead to significant knockdown, region 4 shRNAs (FIG. 2, shRNA 3-6) were all highly active with knockdown of the target gene by almost 90%. The firefly luciferase activity as transfection control showed equal delivery of the transfected plasmids and variation had no correlation with the knockdown values seen (FIG. 3).

Region 4 Constructs Prove Active Against an HIV Integrated Cell Line and a Full HIV Strain

The HlfB cell line is generated by integration of the rev-deficient HXB2 fb HIV strain into HeLa cells. As rev changes the splicing pattern from short mRNAs to long mRNAs which encode the full virus genome and the mRNA encoding the nucleocapsid building protein gag-pol/p24 without rev there is no virus particle formation. Upon transfection of a rev expressing plasmids particle formation can be readily measured by detection of p24 as described above.

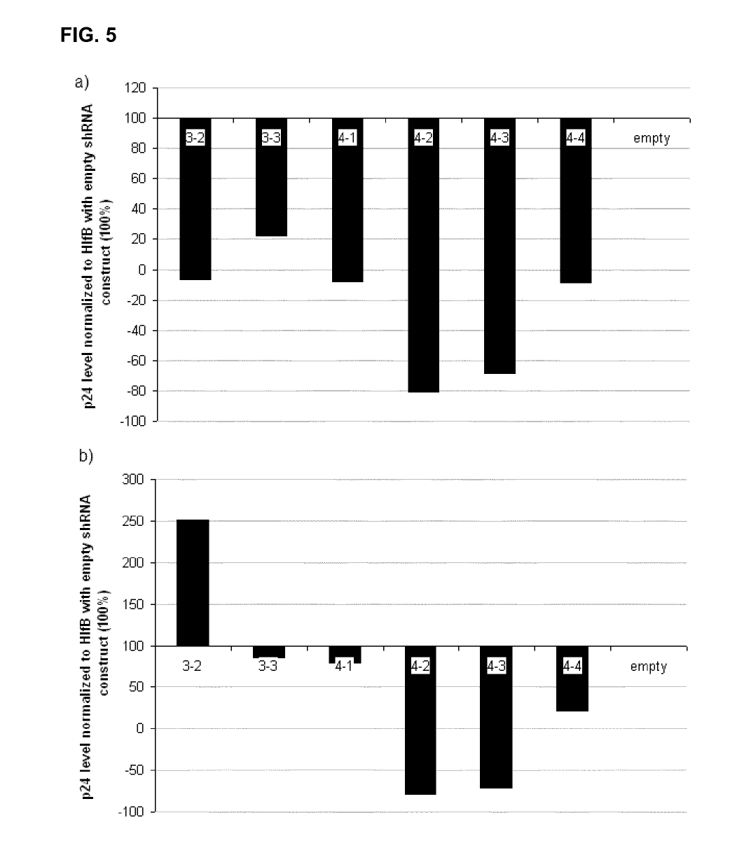

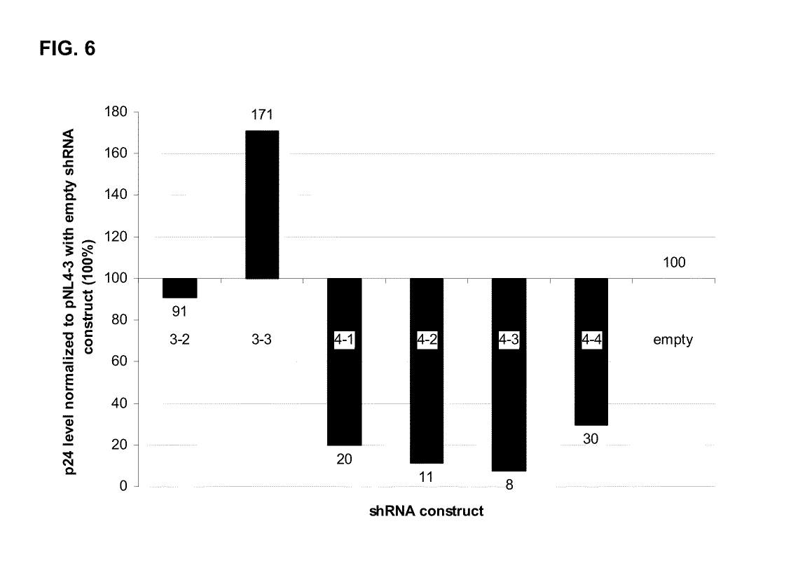

The standard curve for the assay was determined according to manufacturer's protocols and gave a very linear curve with an R(square) of 0.9951 (FIG. 4) at 450 nm wavelength. Strong knockdown was observed with region 4 targeting shRNAs again while region 3 targeting shRNAs showed an effect when 338 ng were transfected indicating a dose response effect (FIG. 5).

Since HlfB is a defective integrated HIV genome needing rev complementation and as it generates a very low p24 level (empty construct transfected gave only a bit over 5 pg p24/ml which is close to the assay detection limit the experiment was repeated with the full HIV strain NL4-3 which can be delivered as provirus encoded in the plasmid pNL4-3. 200 ng of pNL4-3 and 262 ng empty construct was transfected in each sample HEK293T (100.000 cells seeded in 24 well format one day prior transfection). In parallel 338 ng of shRNA or empty construct were transfected as well. 48 hours post transfection p24 levels were measured (same standard curve, FIG. 4).

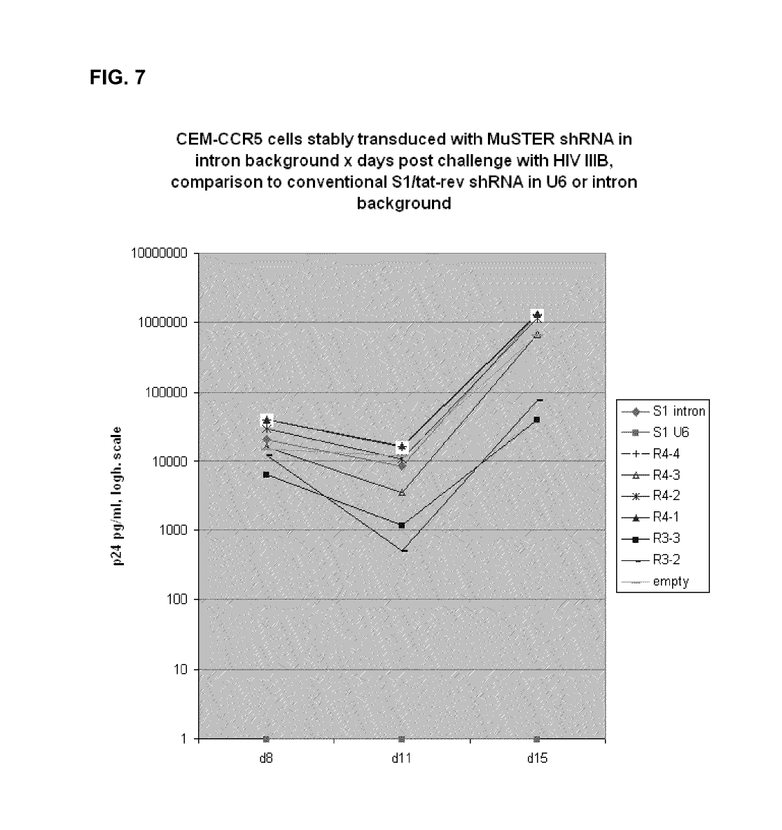

To test whether the dual luciferase results could be confirmed with intact HIV provirus HIV-1 NL4-3 proviral DNA was co-transfected with the MuSTER shRNAs in HEK 293T cells and measured p24 production in the supernatant after 48 hours. The p24 level of NL4-3 was much higher than HlfB and reached 142 pg p24/ml supernatant in the empty sample. Region 3 targeting shRNAs did not cause significant knockdown or up-regulation, while region 4 targeting shRNAs had a strong effect on p24 levels with a maximum knockdown level of 92% (FIG. 6).

A Virus Propagation Assay of HIV IIIB MuSTER Constructs Show Significant Activity in a Stably Transduced CEM-CCR5 T Cell Line

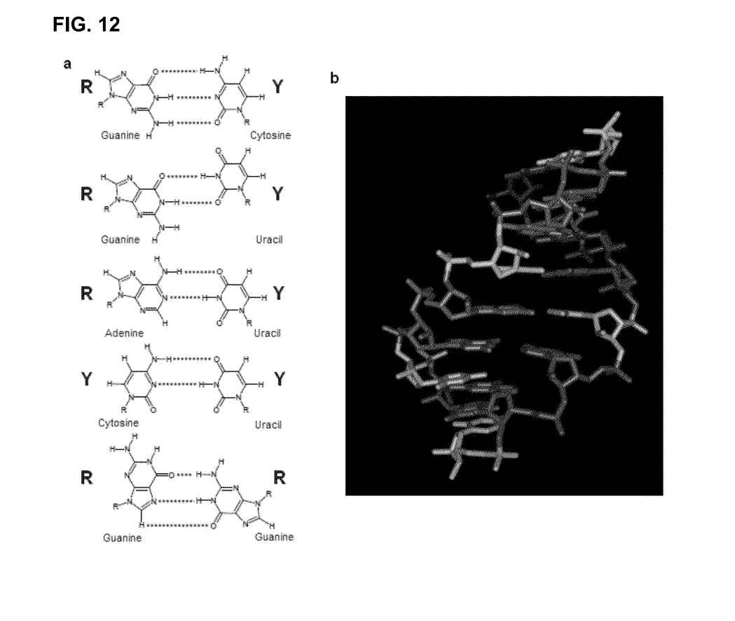

CEM CCR5 cells (obtained from ATCC) were stably transduced with vectors expressing no shRNA, MuSTER shRNA, a conventional shRNA S1 with anti-HIV activity used in Clinical trials in the same background or expressed from the polymerase III U6 promoter. Transduction was determined by GFP expression from the same vector and promoter (except in the case of the U6 driven S1 sequence which was expressed externally of the GFP cassette). CEM cells were transduced with very low multiplier of transduction (MOI) to achieve transduction rates below 10% and thus in majority single copy integration. Cells were sorted for GFP expression and challenged with HIV IIIB virus for 24 hours, next day cells were washed three times to remove remaining virus particles and each 3-4 days until day 11 two third of the volume replaced with fresh media. On day 8, day 11 and d15 supernatant was collected and quantitatively analyzed for p24 expression (Alliance HIV-1 p24 antigen ELISA kit (Perkin Elmer, NEK050001KT) according to manufacturer's protocol) (FIG. 11).

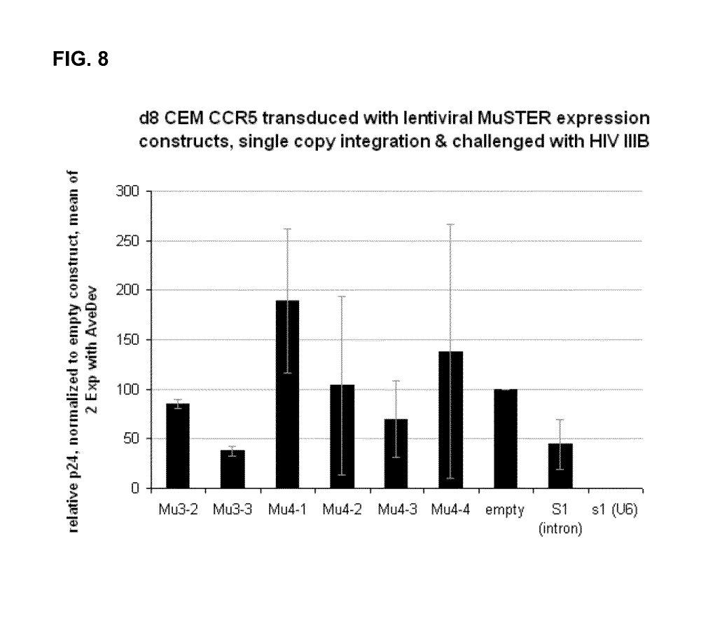

MuSTER designed shRNAs R3-2, R3-3 and R4-3 displayed significant activity against the HIV IIIB replication (FIG. 7). When the highly active U6 driven shRNA S1 was placed in the same background as the MuSTER shRNAs (polymerase II promoter CMV immediate early, which may yield a much lower dose of shRNA than U6) the effect of the conventional and highly active S1 shRNA fell below MuSTER designed R3-2, R3-3 and R4-3. Thus, the MuSTER design shRNAs can yield higher activity than the best known conventional shRNA in the same background.

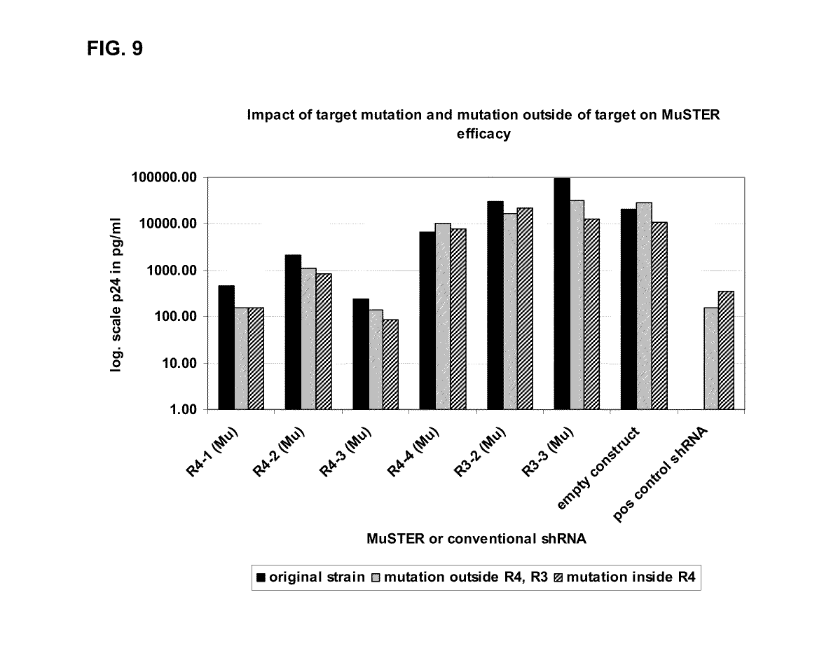

In addition, CEM cells were transduced with CCR5 using a lentiviral MuSTER expression constructs, single copy integration & challenged with HIV IIIB. On day 8, relative p24 expression was measured and normalized to an empty construct, mean of 2 Exp with AveDev. (FIG. 8).

MuSTER Designed shRNAs Retain Activity when Target RNA Undergoes Mutation Inside and Outside of the Target Region

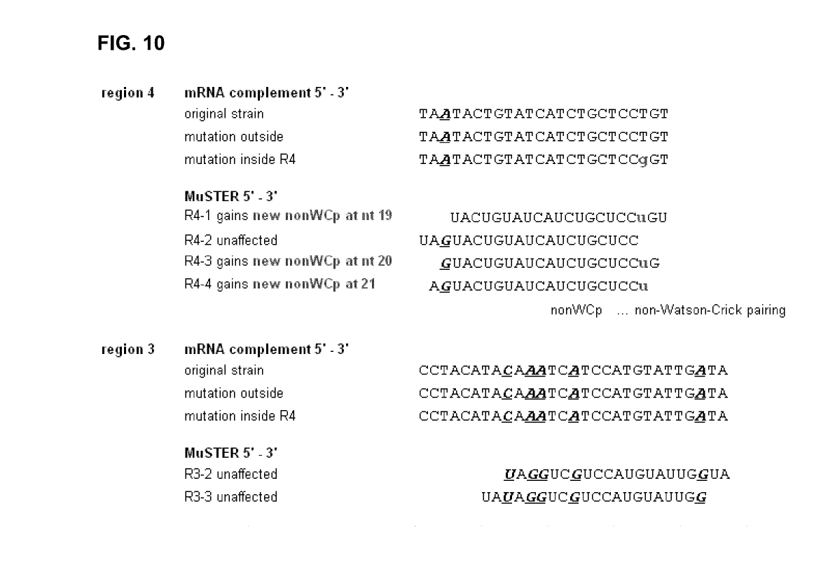

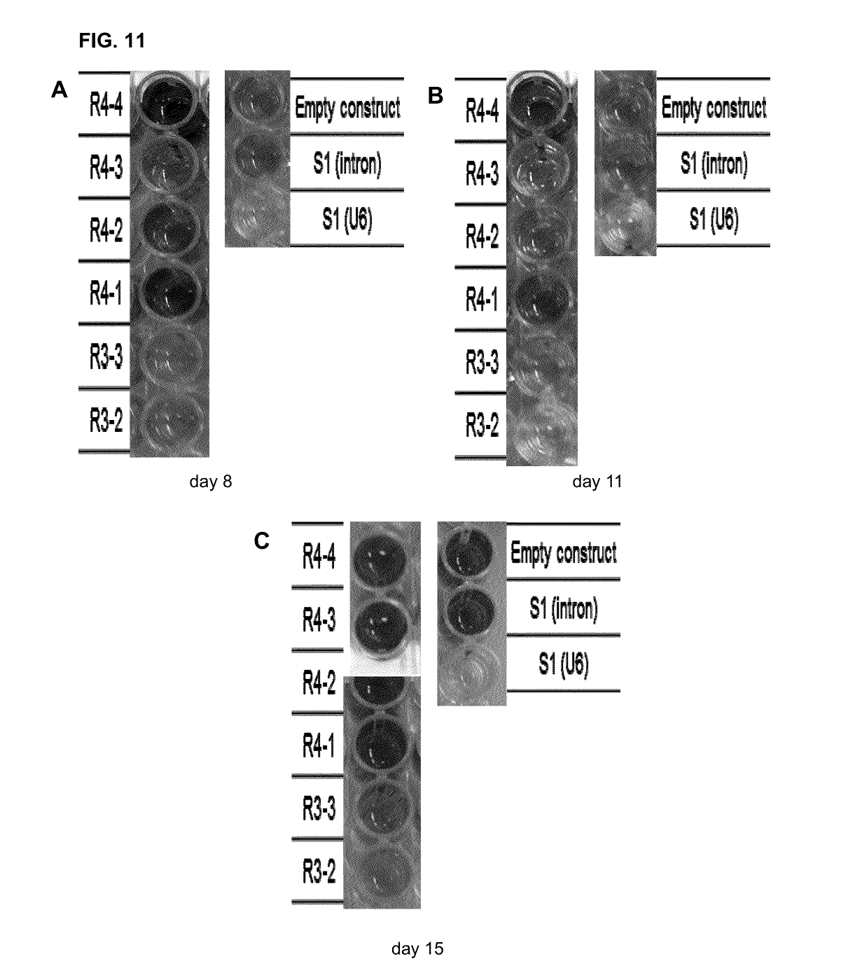

Cells transfected with a plasmid expressing shRNA ID Nos. 3-2, 3-3, 4-1, 4-2, 4-3 and 4-4, an empty construct plasmid (no shRNA or MuSTER-designed shRNA) or a positive control plasmid (conventional shRNA that binds outside of the mutated region) were co-transfected with an original strain and two mutant strains of pNL4-3 HIV provirus. The mutant strains included 1978 nucleotides that span all MuSTER-designed viral RNA targets for two different HIV strains. In one mutant strain, the mutation occurred outside regions 3 and 4, and the second mutant strain included a mutation that occurs inside of region 4. 72 hours after transfection, a p24 assay was performed, the results of which showed that the MuSTER designed shRNAs do not lose their activity when the target viral RNA undergoes further mutation (FIG. 9). The positions and binding between the MuSTER designed shRNAs and the target sequence is shown in FIG. 10. Briefly, shRNA ID No. 4-1 (R4-1) binds region 4 (R4) in the original strain as well as the HIV in the mutant strain with the mutation occurring outside of R4 and regions 3 (R3) with Watson-Crick pairing; but binds the mutant strain with the mutation occurring within R4 with one non-Watson-Crick pair. shRNA ID No. 4-2 (R4-2) binds all three HIV strains within R4 with one non-Watson-Crick pair. shRNA ID No. 4-3 (R4-3) binds all three HIV strains within R4 with one non-Watson-Crick pair, but binds the mutant strain with the mutation occurring within R4 with one additional non-Watson-Crick pair. shRNA ID No. 4-4 (R4-4) binds all three HIV strains within R4 with one non-Watson-Crick pair (at a different position in the guide strand as compared to R4-3), but binds the mutant strain with the mutation occurring within R4 with one additional non-Watson-Crick pair. shRNA ID Nos. 3-2 and 3-3 (R3-2 and R-3-3) bind within R3 in the original strain with several non-Watson-Crick pairings (different positions of the guide strand between both), with no change to the target site in the mutant strains.

Downregulation of Viral Targets by MuSTER shRNAs is Specific and Based on RNAi Mechanisms

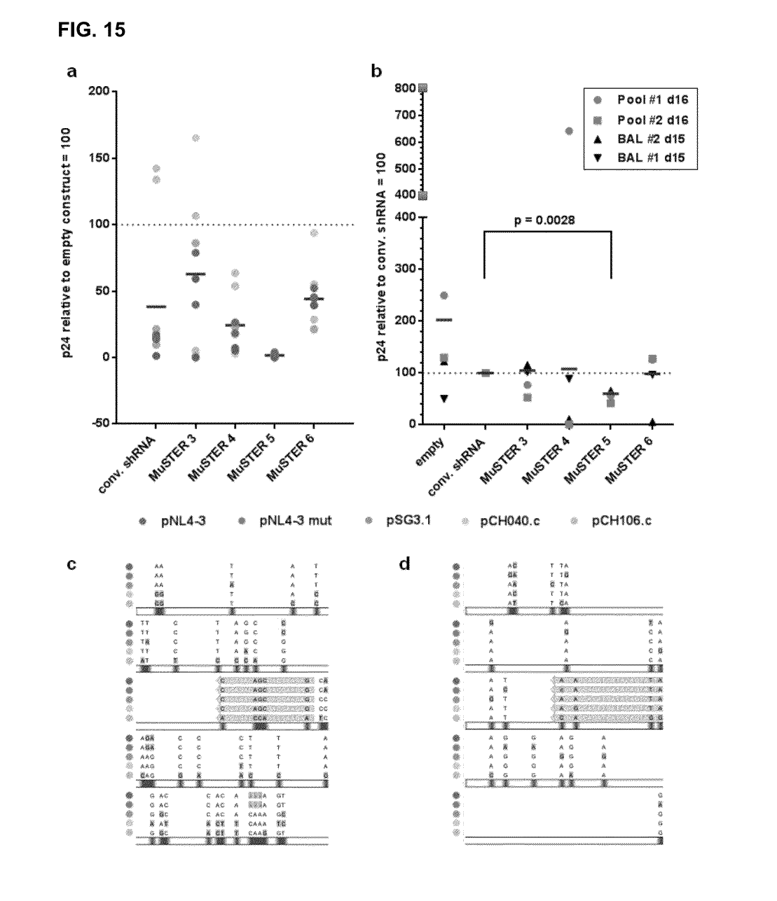

To verify that the MuSTER design was indeed target specific and RNAi based, it was tested whether the resulting shRNAs affected exclusively the only HIV RNA species containing the targeted region—the unspliced variant of HIV mRNA. It was also reasoned that viral inhibition would be lost or decreased when a bulge structure was introduced between guide strand and target RNA (see supplemental files for the complementary bulged MuSTER sequences). p24 assays of the supernatant and Northern Blot analyses of total cellular RNA purified 48 hours post co-transfection of pNL4-3 HIV-1 provirus and shRNA plasmids were performed for MuSTER 4, MuSTER 5, and MuSTER-6 (see FIG. 13) which form nWCp within their target site. The results show a significant p24 reduction for the active MuSTER shRNA constructs and clearly less pronounced effects for the bulged variants (FIG. 14A). Furthermore, as only the unspliced RNA contains the MuSTER target site but all HIV species contain the nef region, probing against nef should yield the full HIV RNA species pattern with all but the unspliced variant unaffected. Northern blot analysis of total cellular RNA from HIV producing cells, which were previously transfected with the MuSTER 4, MuSTER 5, and MuSTER-6 shRNAs, show that the shRNAs are indeed target-specific and reduce only the unspliced form of the HIV mRNA (FIG. 14B). The results also demonstrate that mRNA levels are unaffected for the control bulged constructs, confirming the RNAi mechanism and indicating that the limited p24 protein reductions measured are due to an indirect effect or miRNA-like activity (FIGS. 14A and 14B).

MuSTER Retain their Activity Against Naturally Occurring HIV-1 Variants with Mutation in and Adjacent to the Target Site

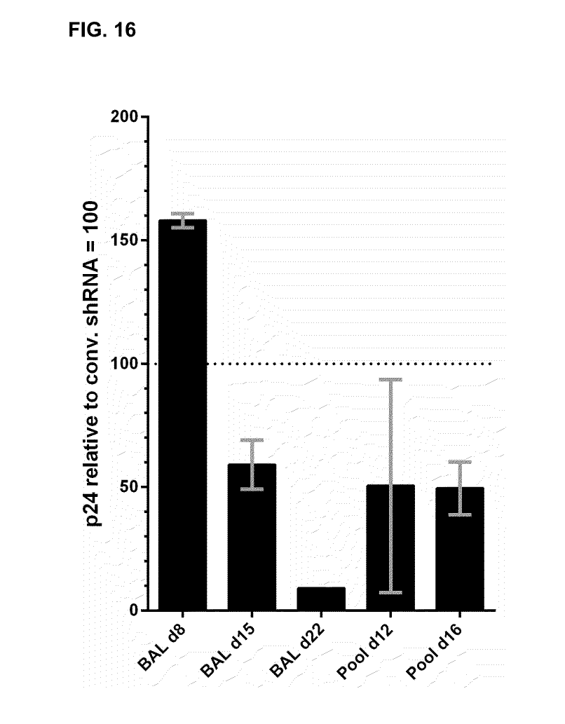

An important aim of the experiments described herein was to retain a robust RNAi activity against multiple naturally found subtypes and variants through the MuSTER design. Screening of Genebank as well as the AIDS repository database of replication competent HIV-1 strains for variant strains yielded very few possible variants in the MuSTER target sites. Every available replication competent strain isolated from patients from the AIDS repository with different target sites was selected and a 1978 nucleotide long stretch of gag-pol in pNL4-3 with the same region from AF005495 replaced to further increase the spectrum of sequence variation against which to challenge the MuSTER design. Cotransfection of the various MuSTER and conventional shRNA plasmids into HEK 293T with the different HIV-1 strains was performed to test whether a single shRNA can be effective against emerging viral mutations. As predicted the conventional control shRNA targeting the tat/rev region of the HIV RNA lost its activity when cotransfected with a HIV-1 strain mutated in the target region. In contrast, MuSTER 5 retained its potent activity in two independent experiments. Indeed MuSTER 5 caused a p24 decrease of over 95% against any HIV-1 variant—which made it more potent than the conventional design even when the later targeted its original target (FIG. 15A).

These previous observations were also verified using a long term infectious assay model system that includes the complete replication cycle as well as mutated targets. To mimic the clinical situation of gene therapy, predominantly single to double genomic integrations of MuSTER expressing cassettes should exert selective pressure against the virus. The previous results should translate into a higher potency of the MuSTER design compared to the conventional potent design from clinical trials when replication competent virus particles infect target cells and are allowed to replicate and thus further mutate for a longer time span.

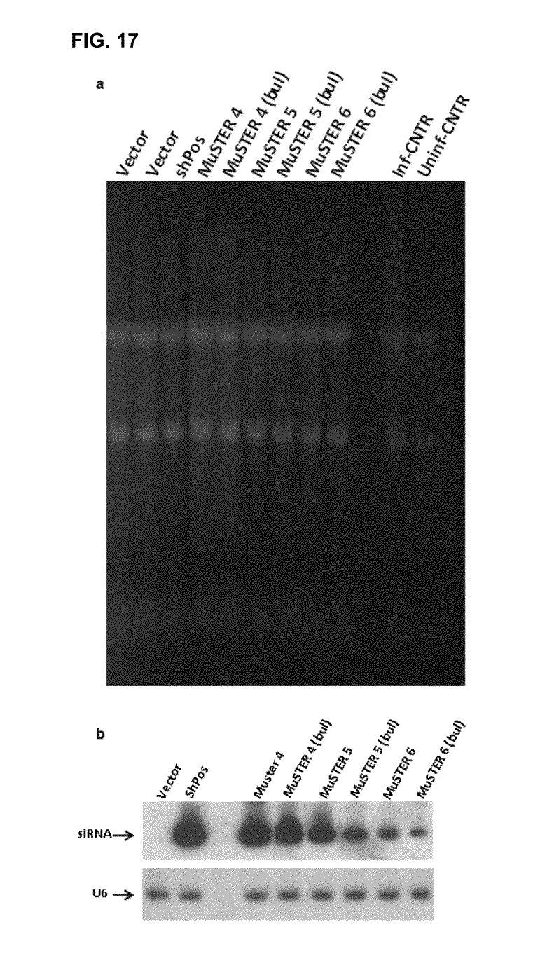

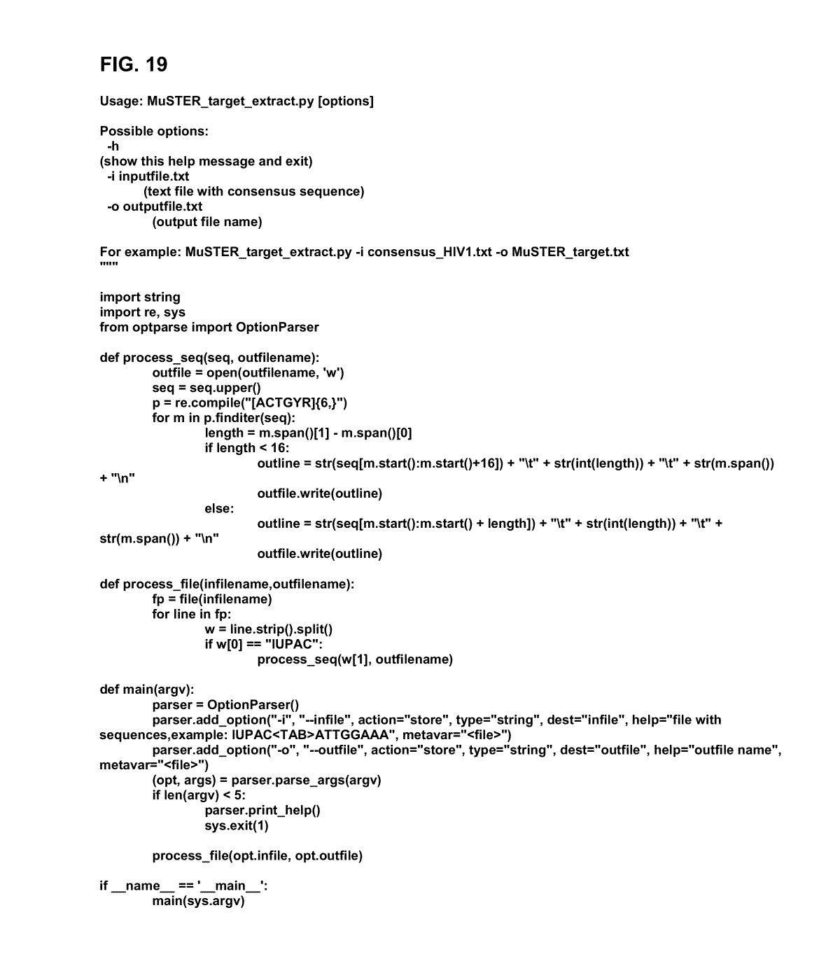

To this end CEM NKR CCR5 T cells were transduced with MuSTER lentiviral vectors, empty vector pHIV8 or a vector with conventional S1 shRNA in the same expression background at a low MOI (transduction rate below 5%, FIG. 18) to achieve mostly single integration events. After cells had been sorted for GFP expression they were challenged with either a HIV-1 BAL III quasi species mix (MOI 0.01, maintained on primary human PBMC) or a pool of the HIV-1 strains used previously in FIG. 15A (this time the fully replicative virus particle supernatant adjusted for same p24 proportions per strain with a total of 2000 pg p24 per 800.000 cells) to mimic emergence of naturally occurring mutations and viral escape. Subsequently, p24 production was analyzed and demonstrated again a significantly higher potency of MuSTER 5 compared to the conventional S1 shRNA. Indeed, MuSTER 5 demonstrated a significantly more potent restriction of both the BAL III quasi species mix as well as the pre-defined mutant pool compared to the conventionally designed highly potent shRNA currently in clinical trials (FIG. 15B). Furthermore this effect improved further or was stable over time compared to the conventional designed shRNA (FIG. 16) proving that the methods underlying the MuSTER design is correct and this novel strategy can improve therapeutic effects against mutating targets.

Discussion

For therapeutic application RNAi is—due to its specificity, low side effects, potency and wide range of potential targets—an attractive tool. This holds especially true if the RNAi trigger is expressed from a genomically integrated cassette as a single treatment could potentially suppress intracellular pathogens like viruses indefinitely. However, when used against polymorphic or mutating genes, common si/shRNA designs, which are exclusively based on canonical Watson and Crick pairing with the corresponding target, have a profound limitation. Since the created mismatches generally cause loss of si/shRNA activity and selective therapeutic pressure in virus infection as well as cancer treatment usually generates escape mutants, therapy is significantly hampered. The same problem occurs in many frequently found infections e.g. with HIV, HCV or human papilloma virus against which even new drugs in clinical trials still only target specific subtypes or clades or risk significant side effects as well as therapeutic cancer targets which tend to exhibit significant escape mutation generation as well. As the problem of escape is a general problem in treatment regimens both with small molecule drugs as well as RNAi it is crucial to develop a new approach, which surmounts this drawback and allows targeting of gene variants. By designing universal sh/siRNAs that incorporate non-canonical base pairing and can tolerate naturally occurring mutations, the problem of escaping RNAi can be overcome.

While HIV RNA has a high flexibility in terms of sequence variation there are still significant mutational constraints due to the necessary encoding of key proteins and the establishment of certain secondary structures. When 54 different HIV-1 strains of a broad range of subtypes and clades from a large global area were aligned, it was found that the genomic RNA shows certain patterns of mutation, with specific nucleotides mutating often only in one or two other bases. Other results support the conclusion that the mutation pattern is not random but reoccurring. Thus, as described above, an RNAi trigger was designed that could offer long term activity even after the emergence of escape variants. This could be achieved by incorporating nWCp in the si/shRNA design that is still capable of complimentary target binding and formation of A-form helical structures with naturally occurring mutated sequences. In other words: the resulting si/shRNA should retain its potency and specificity despite occurrence of target mutation. This new approach is referred to herein as MuSTER (Multi SubType Escape Reducing) design.

As demonstrated above, this is an effective strategy by targeting the HIV gag/pol region with MuSTER shRNAs which establish complementarity with mutant variants through combined WCp and nWCp.

In the experiments described in the Examples above, six shRNAs (see FIG. 13; shRNA ID Nos. 3-2, 3-3, 4-1, 4-2, 4-3, 4-4, also referred to herein as MuSTER 1, 2, 3, 4, 5 and 6) were generated against 2 highly conserved regions in the HIV gag-pol coding sequence. These conserved regions were also found to have many naturally occurring mutants when analyzed against a database containing clinical isolates identified worldwide. Therefore, the shRNAs were designed against artificial IUPAC sequences which take into account all known mutants using a MuSTER method described above. As such, these shRNAs should bind the target region despite mutation as they include non-Watson/Crick base pairing at the position of ambiguity.

While region 3 caused a knockdown effect against HIV strain HxB2 fb with very high shRNA dosage, region 4 targeting shRNAs yielded consistently high knockdown in both HxB2 and NL4-3 strains and the psiCheck reporter. This supports the design strategy since region 4 shRNAs already contains non-Watson/Crick pairing against both strains in case of shRNA ID Nos. 4-2, 4-3 and 4-4 while 4-1 comprises a conventional shRNA.

Conversely, while region 3 targeting shRNAs show no effect in the HIV strains HxB2 and NL4-3 they demonstrate high activity in subsequent experiments with the HIV strain IIIB. The actual target regions in HIV IIIB, HxB2 and NL4-3 should be the same according to sequencing from AIDSrepository.org. But surrounding sequences differ thus folding of surrounding RNA sequences can limit access of si/shRNAs to the individual target sites. This escape mechanism has been shown previously for conventional shRNAs (Westerhout, Ooms et al. 2005) and can be overcome by the combinatorial expression of several si/shRNAs targeting different regions. This approach proves that two different regions both have highly active shRNA of MuSTER design and can cover different HIV strains thus a combinatorial approach of both should cover even mutations that limit accessibility to a single target region.

Another possible explanation for the lower activity of region 4 targeting shRNAs in HIV IIIB would be that this strain not produced from a stable DNA template as is true for NL4-3 and HxB2 but it is produced in a virus propagation assay where virus is used to infect cells which produce fresh virus etc. So HIV IIIB is a pool of several varying sequences and it is not clear how far these might have deviated from the original deposited sequence. They could differ from NL4-3 in the target regions as well beyond the known mutations. This is less likely but can once more be overcome by combinatorial use of MuSTER designed si/shRNAs.

The results from these investigations, which used three HIV strains (HxB2 fb, NL4-3 and IIIB), indicate that the MuSTER designed shRNA of both target regions may have high antiviral activity on their target region with the extend of this effect being impacted either by external sequence structuring or target region mutation. Therefore, in some embodiments, a single RNAi molecule may be used to suppress the expression of the target viral RNA sequence. In other embodiments, a combination of two or more RNAi molecules, each of which bind a different target viral RNA sequence, may be used to suppress the expression of two or more target viral RNA sequences, which would allow protection to be conferred while the virus mutates at least to some degree even outside of the target region.

The potency of these shRNAs was the same or better when compared to a coincidentally canonically fully Watson/Crick pairing shRNA against the same region in that specific target (FIGS. 5 and 6), but, importantly, they also remained functional when challenged with mutant HIV strains (FIG. 15). Furthermore, the effect was stable over time or even improved compared to a conventional potent shRNA (FIG. 16).

As shown by luciferase and p24 assays, the efficacy of the MuSTER shRNAs was reproducible, (FIGS. 2 and 6), and regardless of the genomic integration of the provirus dose dependent (FIG. 5B). Since targeting and degradation occurred only for the unspliced form of the HIV mRNA (containing the shRNA targeted region) and this phenotype was reversed by the introduction of a bulge between target and guide strand (FIG. 14) indicating that the effect was specific. The partial decrease in p24 protein levels uncovered when bulged triggers were used (FIG. 14Aa) is likely to be indirect since there was no visible reduction of the HIV mRNA (FIG. 14B) and perhaps due to titration of RNAi factors such as TRBP or due to suppression of translation in a microRNA-like mechanism. Overall, this strategy allowed the selection of a potent candidate—shRNA MuSTER 5 (also referred to herein as shRNA 4-3)—which possesses all the characteristics to achieve a strong and persistent inhibition against a mutating HIV strain and can possibly overcome the current basic limitation of RNAi therapy.

Furthermore, selecting a target site based on MuSTER rules instead of conventional shRNA design rules has allowed the identification of a novel target site which should be of significant HIV-1 restriction even if the target sequence mutates beyond MuSTER complementarity due to selective pressure. The target region used for the design is highly conserved under MuSTER design rules in wide range of subtypes and clades found all over the world and not B clade restricted. Alignment with strains beyond the initial strain sets have so far not found any naturally occurring sequence that would not be covered by MuSTER 5. This is likely because the target site in the Gag-Pol polyprotein mRNA is downstream of a partially active ribosome frameshift (FIG. 1B). This frameshift regulates the ratio between the upstream single Gag protein (alternative stop codon) and the full read through translation of the Gag-Pol polyprotein (which is further processed upon maturation into Gag and Pol). The thus obtained ratio between Gag and Gag-Pol is important for the fitness of HIV-1 and as long as a MuSTER at least stalls Pol translation (but not the Gag translation upstream of the ribosome frameshift) and thus changes the ratio it should still have a strong detrimental effect on HIV production. That should further favor the MuSTER position for therapy as the secondary structure needed for ribosome shift should restrain mutation of the MuSTER site accessibility at least to some degree. Even if mutations without functional frameshift but changed target site accessibility occur they could still be countered by a combinatorial MuSTER approach. The combination of RNAi triggers is restricted by potential competition between shRNAs as well as perturbation of the endogenous microRNA regulation network. Thanks to the broad specificity of the MuSTER design the number of shRNAs needed for use in combinatorial approaches may be limited or reduced. Another approach could be to multiplex potent MuSTER designs which individually possess less than optimal global sequence variance coverage but can suppress an overall broader target sequences spectrum.

Thus, in some embodiments, the MuSTER design methods and strategy may also be used to predict target accessibility of the HIV strain and compare the results with the MuSTER target stretch selection and chose the precise target region based on MuSTER-compatibility as well as conservation of structure to retain accessibility. Using this strategy, it is possible that MuSTER designed RNAi molecules could be generated that no longer are designed to cover all strains with perfect pairing (Watson Crick and non-Watson Crick/non-canonical) but retain accessibility to the target sequence. Naturally occurring microRNA are active even in case of imperfect complementarity with the target as long as the seed sequence retains perfect pairing. Thus a slightly imperfect pairing beyond the capabilities of non-Watson Crick/non-canonical pairing may still yield highly active sequences and the MuSTER approach is viable even in the case where an shRNA has fewer than 100% pairing with its target sequence (by Watson Crick or non-Watson Crick/non-canonical pairing). A combinatorial construct of several MuSTER-designed shRNA which do not necessarily cover all strains could close the gap to again cover all or the majority of randomly occurring HIV mutants.

The design strategy is also applicable to synthetic siRNAs and antisense oligonucleotides, in which case the variety of possible modification is even larger as it allows for example the introduction of Inosine, selenium bases and modifications found in tRNA anti-codons. Other pairings besides the ones included in this study may be tested in certain positions to expand on potential/frequent mutations. The introduction of si/shRNA design flexibility with these pairings can further be used to adjust RNAi effect strength without having to select another target region.

Since MuSTER based RNAi are standard double stranded small RNAs off target effects should not present any additional issues when compared to a conventional shRNA sequence counterpart. In fact, the occurrence of off target effects may be lower since the possible nWCp with other targets and thus off target binding options have been accounted for. By showing that even non canonical pairings between the guide strand and its target can yield potent decreases in target RNA, these studies raise important questions for currently applied prediction methods to identify potential off target effects. Alternative pairings that could trigger target cleavage or block of translation need to be carefully reconsidered.

In conclusion, the results of the studies described above indicate a strong applicability of nWCp in si/shRNAs without abrogation of the RNAi effect. Due to the novel approach and methods, new RNAi target sites that were previously not considered for RNAi expand the options for designing potent triggers. MuSTER target selection allows for the first time the targeting of highly mutagenic target sequences with a single RNAi trigger. The MuSTER design allows a flexible RNAi trigger to overcome target site mutations seen in infectious diseases and other polymorphic sequences seen in several cancer types of high incidence. Thus this approach should be of great value for many therapy applications. Furthermore the results should have significant impact on the prediction of off target effects of conventional RNAi designs as well as microRNA target site prediction.

### REFERENCES

The references, patents and published patent applications listed below, and all references cited in the specification above are hereby incorporated by reference in their entirety, as if fully set forth herein.
Ameres, S. L. and Zamore, P. D. (2013) Diversifying microRNA sequence and function. Nature reviews. Molecular cell biology, 14, 475-488.
Anderson, A. C., R. H. O'Neil, et al. (1999). “Crystal structure of a brominated RNA helix with four mismatched base pairs: An investigation into RNA conformational variability.” Biochemistry 38(39): 12577-12585.
Anderson, J., Li, M. J., Palmer, B., Remling, L., Li, S., Yam, P., Yee, J. K., Rossi, J., Zaia, J. and Akkina, R. (2007) Safety and efficacy of a lentiviral vector containing three anti-HIV genes—CCR5 ribozyme, tat-rev siRNA, and TAR decoy—in SCID-hu mouse-derived T cells. Mol Ther, 15, 1182-1188.
Bao, Y., P. Bolotov, et al. (2008). “The influenza virus resource at the National Center for Biotechnology Information.” J Virol 82(2): 596-601.
Battle, D. J. and Doudna, J. A. (2002) Specificity of RNA-RNA helix recognition. Proc Natl Acad Sci USA, 99, 11676-11681.
Boden, D., Pusch, O., Lee, F., Tucker, L. and Ramratnam, B. (2003) Human immunodeficiency virus type 1 escape from RNA interference. J Virol, 77, 11531-11535.
Chang, K. C. (2012) Revealing −1 programmed ribosomal frameshifting mechanisms by single-molecule techniques and computational methods. Comput Math Methods Med, 2012, 569870.
Chiu, Y. L. and Rana, T. M. (2002) RNAi in human cells: basic structural and functional features of small interfering RNA. Molecular cell, 10, 549-561.
Das, A. T., Brummelkamp, T. R., Westerhout, E. M., Vink, M., Madiredjo, M., Bernards, R. and Berkhout, B. (2004) Human immunodeficiency virus type 1 escapes from RNA interference-mediated inhibition. J Virol, 78, 2601-2605.
DiGiusto, D. L., Krishnan, A., Li, L., Li, H., Li, S., Rao, A., Mi, S., Yam, P., Stinson, S., Kalos, M. et al. (2010) RNA-based gene therapy for HIV with lentiviral vector-modified CD34(+) cells in patients undergoing transplantation for AIDS-related lymphoma. Sci Transl Med, 2, 36ra43.
Doench, J. G., Petersen, C. P. and Sharp, P. A. (2003) siRNAs can function as miRNAs. Genes Dev, 17, 438-442.
Du, G., Yonekubo, J., Zeng, Y., Osisami, M. and Frohman, M. A. (2006) Design of expression vectors for RNA interference based on miRNAs and RNA splicing. FEBS J, 273, 5421-5427.
Du, Q., Thonberg, H., Wang, J., Wahlestedt, C. and Liang, Z. (2005) A systematic analysis of the silencing effects of an active siRNA at all single-nucleotide mismatched target sites. Nucleic Acids Res, 33, 1671-1677.
Finnegan, E. F. and Pasquinelli, A. E. (2013) MicroRNA biogenesis: regulating the regulators. Critical reviews in biochemistry and molecular biology, 48, 51-68.
Holen, T., Moe, S. E., Sorbo, J. G., Meza, T. J., Ottersen, O. P. and Klungland, A. (2005) Tolerated wobble mutations in siRNAs decrease specificity, but can enhance activity in vivo. Nucleic Acids Res, 33, 4704-4710.
Joseph, T. T. and Osman, R. (2012) Convergent transmission of RNAi guide-target mismatch information across Argonaute internal allosteric network. PLoS Comput Biol, 8, e1002693.
Kagita, S., Uppalapati, S., Jiwatani, S., Linga, V. G., Gundeti, S., Nagesh, N. and Digumarti, R. (2014) Incidence of Bcr-Abl kinase domain mutations in imatinib refractory chronic myeloid leukemia patients from South India. Tumour biology: the journal of the International Society for Oncodevelopmental Biology and Medicine.
Kiliszek, A., Kierzek, R., Krzyzosiak, W. J. and Rypniewski, W. (2011) Crystal structures of CGG RNA repeats with implications for fragile X-associated tremor ataxia syndrome. Nucleic Acids Res, 39, 7308-7315.
Li, M. J., Bauer, G., Michienzi, A., Yee, J. K., Lee, N. S., Kim, J., Li, S., Castanotto, D., Zaia, J. and Rossi, J. J. (2003) Inhibition of HIV-1 infection by lentiviral vectors expressing Pol III-promoted anti-HIV RNAs. Mol Ther, 8, 196-206.
Li, M. J., Kim, J., Li, S., Zaia, J., Yee, J. K., Anderson, J., Akkina, R. and Rossi, J. J. (2005) Long-term inhibition of HIV-1 infection in primary hematopoietic cells by lentiviral vector delivery of a triple combination of anti-HIV shRNA, anti-CCR5 ribozyme, and a nucleolar-localizing TAR decoy. Mol Ther, 12, 900-909.
Lisowski, L., Elazar, M., Chu, K., Glenn, J. S, and Kay, M. A. (2013) The anti-genomic (negative) strand of Hepatitis C Virus is not targetable by shRNA. Nucleic Acids Res, 41, 3688-3698.
Mermer, B., Felber, B. K., Campbell, M. and Pavlakis, G. N. (1990) Identification of trans-dominant HIV-1 rev protein mutants by direct transfer of bacterially produced proteins into human cells. Nucleic Acids Res, 18, 2037-2044.
Messina, M., Del Giudice, I., Khiabanian, H., Rossi, D., Chiaretti, S., Rasi, S., Spina, V., Holmes, A. B., Marinelli, M., Fabbri, G. et al. (2014) Genetic lesions associated with chronic lymphocytic leukemia chemo-refractoriness. Blood, 123, 2378-2388.
Panigrahi, S., Pal, R. and Bhattacharyya, D. (2011) Structure and energy of non-canonical basepairs: comparison of various computational chemistry methods with crystallographic ensembles. Journal of biomolecular structure & dynamics, 29, 541-556.
Rana, T. M. (2007) Illuminating the silence: understanding the structure and function of small RNAs. Nature reviews. Molecular cell biology, 8, 23-36.
Sanghvi, V. R. and Steel, L. F. (2011) The cellular TAR RNA binding protein, TRBP, promotes HIV-1 replication primarily by inhibiting the activation of double-stranded RNA-dependent kinase PKR. J Virol, 85, 12614-12621.
Schaub, M. and Keller, W. (2002) RNA editing by adenosine deaminases generates RNA and protein diversity. Biochimie, 84, 791-803.
Scherer, L. J., Yildiz, Y., Kim, J., Gagnon, L., Heale, B. and Rossi, J. J. (2004) Rapid assessment of anti-HIV siRNA efficacy using PCR-derived Pol III shRNA cassettes. Mol Ther, 10, 597-603.
Schwartz, S., Felber, B. K. and Pavlakis, G. N. (1992) Distinct RNA sequences in the gag region of human immunodeficiency virus type 1 decrease RNA stability and inhibit expression in the absence of Rev protein. J Virol, 66, 150-159.
Schwarz, D. S., Ding, H., Kennington, L., Moore, J. T., Schelter, J., Burchard, J., Linsley, P. S., Aronin, N., Xu, Z. and Zamore, P. D. (2006) Designing siRNA that distinguish between genes that differ by a single nucleotide. PLoS Genet, 2, e140.
Shehu-Xhilaga, M., Crowe, S. M. and Mak, J. (2001) Maintenance of the Gag/Gag-Pol ratio is important for human immunodeficiency virus type 1 RNA dimerization and viral infectivity. J Virol, 75, 1834-1841.
Stedman, C. A. (2013) Current prospects for interferon-free treatment of hepatitis C in 2012. Journal of gastroenterology and hepatology, 28, 38-45.
Sun, H., Sheng, J., Hassan, A. E., Jiang, S., Gan, J. and Huang, Z. (2012) Novel RNA base pair with higher specificity using single selenium atom. Nucleic Acids Res.
ter Brake, O., von Eije, K. J. and Berkhout, B. (2008) Probing the sequence space available for HIV-1 evolution. AIDS, 22, 1875-1877.
Tiemann, K. and Rossi, J. J. (2009) RNAi-based therapeutics-current status, challenges and prospects. EMBO molecular medicine, 1, 142-151.
Varani, G. and McClain, W. H. (2000) The G×U wobble base pair. A fundamental building block of RNA structure crucial to RNA function in diverse biological systems. EMBO Rep, 1, 18-23.
Vendeix, F. A., Murphy, F. V. t., Cantara, W. A., Leszczynska, G., Gustilo, E. M., Sproat, B., Malkiewicz, A. and Agris, P. F. (2012) Human tRNA(Lys3)(UUU) is pre-structured by natural modifications for cognate and wobble codon binding through keto-enol tautomerism. J Mol Biol, 416, 467-485.
Vert, J. P., Foveau, N., Lajaunie, C. and Vandenbrouck, Y. (2006) An accurate and interpretable model for siRNA efficacy prediction. BMC bioinformatics, 7, 520.
Westerhout, E. M., Ooms, M., Vink, M., Das, A. T. and Berkhout, B. (2005) HIV-1 can escape from RNA interference by evolving an alternative structure in its RNA genome. Nucleic Acids Res, 33, 796-804.
Wilson, J. A. and Richardson, C. D. (2005) Hepatitis C virus replicons escape RNA interference induced by a short interfering RNA directed against the NS5b coding region. J Virol, 79, 7050-7058.

What is claimed is:

1. A universal nucleic acid binding molecule comprising:
an inhibitory or activating nucleic acid sequence which binds a receiving nucleic acid sequence, via at least one non-Watson Crick or non-canonical paired base.

2. The universal nucleic acid binding molecule of claim 1, wherein the receiving nucleic acid sequence is a coding RNA sequence, a coding DNA sequence, a non-coding RNA sequence, or a non-coding DNA sequence,

3. The RNAi molecule of claim 2, wherein the receiving nucleic acid sequence is (i) a viral RNA sequence derived from a human immunodeficiency HIV virus, a hepatitis B virus (HBV), a hepatitis C virus (HCV), or an influenza virus; or (ii) a DNA or RNA sequence derived from an oncogene or variant thereof that is associated with the development of cancer, chemotherapy resistance, or both.

4. The universal nucleic acid binding molecule of claim 3, wherein the target viral RNA sequence comprises an IUPAC sequence of
(SEQ ID NO: 1)
URYCARUAYAUGGAYGAYYURUAUGURGG

5. The universal nucleic acid binding molecule of claim 4, wherein the inhibitory or activating nucleic acid sequence comprises an IUPAC sequence selected from the group consisting of
(SEQ ID NO: 3)
YARRTCRTCCATRTAYTGRYA;
or
(SEQ ID NO: 4)
TAYARRTCRTCCATRTAYTGR

6. The universal nucleic acid binding molecule of claim 5, wherein the inhibitory or activating nucleic acid sequence comprises a sequence selected from
(SEQ ID NO: 5)
TAGGTCGTCCATGTATTGGTA;
or
(SEQ ID NO: 6)
TATAGGTCGTCCATGTATTGG.

7. The universal nucleic acid binding molecule of claim 3, wherein the target viral RNA sequence comprises an IUPAC sequence of
(SEQ ID NO: 2)
YURGAYACRGGRGCAGAUGAUACAGUR.

8. The universal nucleic acid binding molecule of claim 7, wherein the inhibitory or activating nucleic acid sequence comprises an IUPAC sequence selected from the group consisting of
(SEQ ID NO: 7)
YACTGTATCATCTGCYCCYGT;
(SEQ ID NO: 8)
YADYACTGTATCATCTGCYCC;
(SEQ ID NO: 9)
DYACTGTATCATCTGCYCCYG;
and
(SEQ ID NO: 10)
ADYACTGTATCATCTGCYCCY.

9. The universal nucleic acid binding molecule of claim 8, wherein the inhibitory or activating nucleic acid sequence comprises a sequence selected from
(SEQ ID NO: 11)
TACTGTATCATCTGCTCCTGT;
(SEQ ID NO: 12)
TAGTACTGTATCATCTGCTCC;
(SEQ ID NO: 13)
GTACTGTATCATCTGCTCCTG;
and
(SEQ ID NO: 14)
AGTACTGTATCATCTGCTCCT.

10. A method of treating a subject having a disease or condition comprising administering a therapeutically effective amount of a universal nucleic acid binding molecule which comprises an inhibitory or activating nucleic acid sequence which binds a receiving nucleic acid sequence via at least one non-Watson Crick or non-canonical paired base; wherein the universal nucleic acid binding molecule activates or suppresses the expression or activity of a target molecule.

11. The method of claim 10, wherein the subject is infected with a target virus, the disease or condition is a viral infection, and the receiving nucleic acid sequence is a viral RNA sequence is derived from a human immunodeficiency HIV virus, a hepatitis C virus (HCV), a hepatitis B virus (HBV), or an influenza virus.

12. The method of claim 11, wherein the target virus is a human immunodeficiency HIV virus.

13. The method of claim 12, wherein the target viral RNA sequence comprises an IUPAC sequence of URYCARUAYAUGGAYGAYYURUAUGURGG (SEQ ID NO:1)

14. The method of claim 13, wherein the inhibitory or activating nucleic acid sequence comprises an IUPAC sequence selected from the group consisting of
(SEQ ID NO: 3)
YARRTCRTCCATRTAYTGRYA;
or
(SEQ ID NO: 4)
TAYARRTCRTCCATRTAYTGR

15. The method of claim 13, wherein the inhibitory or activating nucleic acid sequence comprises a sequence selected from
(SEQ ID NO: 5)
TAGGTCGTCCATGTATTGGTA;
or
(SEQ ID NO: 6)
TATAGGTCGTCCATGTATTGG.

16. The method of claim 12, wherein the target viral RNA sequence comprises an IUPAC sequence of YURGAYACRGGRGCAGAUGAUACAGUR (SEQ ID NO:2).

17. The method of claim 16, wherein the inhibitory or activating nucleic acid sequence comprises an IUPAC sequence selected from the group consisting of
(SEQ ID NO: 7)
YACTGTATCATCTGCYCCYGT;
(SEQ ID NO: 8)
YADYACTGTATCATCTGCYCC;
(SEQ ID NO: 9)
DYACTGTATCATCTGCYCCYG;
and
(SEQ ID NO: 10)
ADYACTGTATCATCTGCYCCY.

18. The method of claim 16, wherein the inhibitory or activating nucleic acid sequence comprises a sequence selected from
(SEQ ID NO: 11)
TACTGTATCATCTGCTCCTGT;
(SEQ ID NO: 12)
TAGTACTGTATCATCTGCTCC;
(SEQ ID NO: 13)
GTACTGTATCATCTGCTCCTG;
and
(SEQ ID NO: 14)
AGTACTGTATCATCTGCTCCT.

19. A method of designing a universal nucleic acid binding molecule comprising generating an inhibitory or activating nucleic acid sequence by
(i) generating a consensus sequence derived from two or more receiving nucleic acid sequences of host or foreign origin and converting the consensus sequence into an International Union of Pure and Applied Chemistry (IUPAC) sequence code by randomly or selectively replacing U or G's with a corresponding ambiguous IUPAC coded base (Y or R); or
(ii) generating an artificial target sequence from one target nucleic acid sequence of host of foreign origin by randomly or selectively replacing U or G's with a corresponding ambiguous IUPAC coded base (Y or R);
substituting each corresponding ambiguous base with a base that allows for Watson-Crick, non-Watson-Crick, and non-canonical pairings; and
selecting one or more inhibitory RNA molecules using an RNAi selection method, an antisense oligonucleotide selection protocol, or an RNA-based transcriptional activation or inhibition selection method.

20. The method of claim 19, wherein the consensus sequence is derived from a plurality of clinical isolates.


SEQUENCE LISTING (62 sequences)

SEQ ID NO: 1 - RNA, 29 nt, Artificial Sequence
  Synthetic oligonucleotide
urycaruayauggaygayyuruaugurgg

SEQ ID NO: 2 - RNA, 27 nt, Artificial Sequence
  Synthetic oligonucleotide
yurgayacrggrgcagaugauacagur

SEQ ID NO: 3 - DNA, 21 bp, Artificial Sequence
  Synthetic oligonucleotide
yarrtcrtccatrtaytgrya

SEQ ID NO: 4 - DNA, 21 bp, Artificial Sequence
  Synthetic oligonucleotide
tayarrtcrtccatrtaytgr

SEQ ID NO: 5 - DNA, 21 bp, Artificial Sequence
  Synthetic oligonucleotide
taggtcgtccatgtattggta

SEQ ID NO: 6 - DNA, 21 bp, Artificial Sequence
  Synthetic oligonucleotide
tataggtcgtccatgtattgg

SEQ ID NO: 7 - DNA, 21 bp, Artificial Sequence
  Synthetic oligonucleotide
yactgtatcatctgcyccygt

SEQ ID NO: 8 - DNA, 21 bp, Artificial Sequence
  Synthetic oligonucleotide
yadyactgtatcatctgcycc

SEQ ID NO: 9 - DNA, 21 bp, Artificial Sequence
  Synthetic oligonucleotide
dyactgtatcatctgcyccyg

SEQ ID NO: 10 - DNA, 21 bp, Artificial Sequence
  Synthetic oligonucleotide
adyactgtatcatctgcyccy

SEQ ID NO: 11 - DNA, 21 bp, Artificial Sequence
  Synthetic oligonucleotide
tactgtatcatctgctcctgt

SEQ ID NO: 12 - DNA, 21 bp, Artificial Sequence
  Synthetic oligonucleotide
tagtactgtatcatctgctcc

SEQ ID NO: 13 - DNA, 21 bp, Artificial Sequence
  Synthetic oligonucleotide
gtactgtatcatctgctcctg

SEQ ID NO: 14 - DNA, 21 bp, Artificial Sequence
  Synthetic oligonucleotide
agtactgtatcatctgctcct

SEQ ID NO: 15 - DNA, 71 bp, Artificial Sequence
  Synthetic oligonucleotide
agcgataccaatacatggacgacctatagtgaagccacagatgtataggtcgtccatgtattggtagtgcc

SEQ ID NO: 16 - DNA, 71 bp, Artificial Sequence
  Synthetic oligonucleotide
agcggccaatacatggacgacctatatagtgaagccacagatgtatataggtcgtccatgtattggttgcc

SEQ ID NO: 17 - DNA, 71 bp, Artificial Sequence
  Synthetic oligonucleotide
agcgaacaggagcagatgatacagtatagtgaagccacagatgtatactgtatcatctgctcctgtgtgcc

SEQ ID NO: 18 - DNA, 71 bp, Artificial Sequence
  Synthetic oligonucleotide
agcggggagcagatgatacagtactatagtgaagccacagatgtatagtactgtatcatctgctccttgcc

SEQ ID NO: 19 - DNA, 71 bp, Artificial Sequence
  Synthetic oligonucleotide
agcggcaggagcagatgatacagtactagtgaagccacagatgtagtactgtatcatctgctcctgttgcc

SEQ ID NO: 20 - DNA, 71 bp, Artificial Sequence
  Synthetic oligonucleotide
agcgaaggagcagatgatacagtacttagtgaagccacagatgtaagtactgtatcatctgctcctgtgcc

SEQ ID NO: 21 - RNA, 32 nt, Artificial Sequence
  Synthetic oligonucleotide
arruggagraaryurgurgayuuyagrgaryu

SEQ ID NO: 22 - RNA, 28 nt, Artificial Sequence
  Synthetic oligonucleotide
raugayaucacaraaryurgurggraar

SEQ ID NO: 23 - RNA, 22 nt, Artificial Sequence
  Synthetic oligonucleotide
aararrggyugyurgaarugur

SEQ ID NO: 24 - RNA, 22 nt, Artificial Sequence
  Synthetic oligonucleotide
rrgggarrragaurrgugcrag

SEQ ID NO: 25 - RNA, 21 nt, Artificial Sequence
  Synthetic oligonucleotide
uaggucguccauguauuggua

SEQ ID NO: 26 - DNA, 21 bp, Artificial Sequence
  Synthetic oligonucleotide
gtttagtaggtacataactat

SEQ ID NO: 27 - RNA, 21 nt, Artificial Sequence
  Synthetic oligonucleotide
uauaggucguccauguauugg

SEQ ID NO: 28 - DNA, 21 bp, Artificial Sequence
  Synthetic oligonucleotide
atgtttagtaggtacataact

SEQ ID NO: 29 - RNA, 21 nt, Artificial Sequence
  Syntheitc oligonucleotide
uacuguaucaucugcuccugu

SEQ ID NO: 30 - DNA, 21 bp, Artificial Sequence
  Syntheitc oligonucleotide
atgacatagtagacgaggaca

SEQ ID NO: 31 - RNA, 21 nt, Artificial Sequence
  Syntheitc oligonucleotide
uaguacuguaucaucugcucc

SEQ ID NO: 32 - DNA, 21 bp, Artificial Sequence
  Syntheitc oligonucleotide
attatgacatagtagacgagg

SEQ ID NO: 33 - RNA, 21 nt, Artificial Sequence
  Syntheitc oligonucleotide
guacuguaucaucugcuccug

SEQ ID NO: 34 - DNA, 21 bp, Artificial Sequence
  Syntheitc oligonucleotide
tatgacatagtagacgaggac

SEQ ID NO: 35 - RNA, 21 nt, Artificial Sequence
  Syntheitc oligonucleotide
aguacuguaucaucugcuccu

SEQ ID NO: 36 - DNA, 21 bp, Artificial Sequence
  Syntheitc oligonucleotide
ttatgacatagtagacgagga

SEQ ID NO: 37 - DNA, 27 bp, Artificial Sequence
  Syntheitc oligonucleotide
ytrgayacrggrgcagatgatacagtr

SEQ ID NO: 38 - DNA, 30 bp, Artificial Sequence
  Syntheitc oligonucleotide
atatctcgagcttcagagcagaccagagcc

SEQ ID NO: 39 - DNA, 32 bp, Artificial Sequence
  Syntheitc oligonucleotide
tatagcggccgctccccacctcaacagatgtt

SEQ ID NO: 40 - DNA, 29 bp, Artificial Sequence
  Synthetic oligonucleotide
trycartayatggaygayytrtatgtrgg

SEQ ID NO: 41 - DNA, 30 bp, Artificial Sequence
  Synthetic oligonucleotide
ggaagtgatatagctggaactactagtacc

SEQ ID NO: 42 - DNA, 37 bp, Artificial Sequence
  Synthetic oligonucleotide
atatattccggatcttttagaatctccctgttttctg

SEQ ID NO: 43 - DNA, 69 bp, Artificial Sequence
  Synthetic oligonucleotide
cgcgactctagatcatggatcctccggactgcactctagataggtcaccaccgtcgactagccgtacct

SEQ ID NO: 44 - DNA, 22 bp, Artificial Sequence
  Synthetic oligonucleotide
actatagggcgaattgggtacc

SEQ ID NO: 45 - DNA, 43 bp, Artificial Sequence
  Synthetic oligonucleotide
tattcgcactggatacgatccggatgattctgtggataaccgt

SEQ ID NO: 46 - DNA, 46 bp, Artificial Sequence
  Synthetic oligonucleotide
aatatcctccttagttccggtgacctagaatgcagtgaaaaaaatg

SEQ ID NO: 47 - DNA, 37 bp, Artificial Sequence
  Synthetic oligonucleotide
gccatcgatattgttagctgctgtattgctacttgtg

SEQ ID NO: 48 - DNA, 21 bp, Artificial Sequence
  Synthetic oligonucleotide
gcggagacagcgacgaagagc

SEQ ID NO: 49 - DNA, 22 bp, Artificial Sequence
  Synthetic oligonucleotide
tatggaacgcttcacgaatttg

SEQ ID NO: 50 - DNA, 22 bp, Artificial Sequence
  Synthetic oligonucleotide
aggagcagatgatacagtacta

SEQ ID NO: 51 - DNA, 23 bp, Artificial Sequence
  Synthetic oligonucleotide
aggagcagtaacatacagtacta

SEQ ID NO: 52 - DNA, 22 bp, Artificial Sequence
  Synthetic oligonucleotide
acaggagcagatgatacagtac

SEQ ID NO: 53 - DNA, 23 bp, Artificial Sequence
  Synthetic oligonucleotide
acaggagctccttgatacagtac

SEQ ID NO: 54 - DNA, 22 bp, Artificial Sequence
  Synthetic oligonucleotide
caggagcagatgatacagtact

SEQ ID NO: 55 - DNA, 23 bp, Artificial Sequence
  Synthetic oligonucleotide
caggagcacttagatacagtact

SEQ ID NO: 56 - DNA, 72 bp, Artificial Sequence
  Synthetic oligonucleotide
agcgataccaatacatggacgacctatagtgaagccacagatgtataggtcgtccaaccaattggtagtgcc

SEQ ID NO: 57 - DNA, 72 bp, Artificial Sequence
  Synthetic oligonucleotide
agcggccaatacatggacgacctatatagtgaagccacagatgtatataggtcgtcgttagtattggttgcc

SEQ ID NO: 58 - DNA, 72 bp, Artificial Sequence
  Synthetic oligonucleotide
agcgaacaggagcagatgatacagtatagtgaagccacagatgtatactgtatcatgaacctcctgtgtgcc

SEQ ID NO: 59 - DNA, 72 bp, Artificial Sequence
  Synthetic oligonucleotide
agcggggagcagatgatacagtactatagtgaagccacagatgtatagtactgtatgttactgctccttgcc

SEQ ID NO: 60 - DNA, 72 bp, Artificial Sequence
  Synthetic oligonucleotide
agcggcaggagcagatgatacagtactagtgaagccacagatgtagtactgtatcaaggagctcctgttgcc

SEQ ID NO: 61 - DNA, 72 bp, Artificial Sequence
  Synthetic oligonucleotide
agcgaaggagcagatgatacagtacttagtgaagccacagatgtaagtactgtatctaagtgctcctgtgcc

SEQ ID NO: 62 - DNA, 71 bp, Artificial Sequence
  Synthetic oligonucleotide
agcgcgcggagacagcgacgaagagctagtgaagccacagatgtagctcttcgtcgctgtctccgcttgcc